(19) United States
(12) Patent Application Publication
(10) Pub. No.: US 2025/0288451 A1
(43) Pub. Date: Sep. 18, 2025

(54) Artificial intestinal device and surgical method using the same

(71) Applicant: Entax Biomedicals CO., LTD., New Taipei City (TW)

(72) Inventors: Chien-Chang Lu, New Taipei City (TW); Paichun Chang, New Taipei City (TW); Ming-Yuan Tsai, New Taipei City (TW)

(21) Appl. No.: 19/068,278

(22) Filed: Mar. 3, 2025

Related U.S. Application Data
(60) Provisional application No. 63/565,262, filed on Mar. 14, 2024.

(51) Int. Cl. A61F 5/449; A61F 5/445

(52) U.S. Cl. CPC A61F 5/449; A61F 2005/4455

(57) ABSTRACT

A surgical method comprises following steps of: making an incision of an abdominal wall; making a first-incision of an ileum near a terminal ileum; passing an assembly into the ileum through the first-incision; making a second-incision of the ileum; pulling a tip-portion of a guiding-member of the assembly out of the ileum through the second-incision such that an inflated-collector-portion of an artificial-ileal-tube of the assembly is stuck at the second-incision; suturing the first-incision; passing one end of a fixing-tube through a mesentery behind the ileum for being suspended beneath the ileum; making a for-tube-incision and a for-fixing-incision of the abdominal wall; passing the guiding-member and two ends of the fixing-tube out of the abdominal wall through the for-tube-incision and the for-fixing-incision respectively; fixing the fixing-tube; suturing the incision of the abdominal wall; detaching the guiding-member from the artificial-ileal-tube; and connecting a collecting device to the artificial-ileal-tube.

### CROSS-REFERENCE TO RELATED DOCUMENTS

The present invention claims the benefit of U.S. Provisional Patent Application No. 63/565,262, filed on Mar. 14, 2024, the disclosure of which is incorporated herein by reference in its entirety.

### FIELD OF THE INVENTION

The present invention relates to an artificial intestinal device and a surgical method using thereof, especially an artificial intestinal device with an inflated collector portion for collecting intestinal fluid.

### BACKGROUND OF THE INVENTION

The creation of an intestinal stoma is an integral part of many surgeries, such as the diverticulitis of the colon, colon cancer and rectal cancer. Conventional surgical method is to open an abdomen of a patient by a laparotomy, and then to make an incision of an abdominal wall of the patient for creating an intestinal stoma. After cutting an ileum such that two openings of the ileum are formed, wherein one opening (an active opening) of the two openings of the ileum is not in communication with a cecum; while the other one opening (a distal opening) of the two openings of the ileum is in communication with the cecum. At least the active opening of the ileum is pulled out of the abdominal wall of the patient through the incision of the abdominal wall and then a surrounding portion of the ileum around the active opening of the ileum is secured on an outer surface of the abdominal wall to create an ileostomy. Then the ileostomy is expanded by a tool and the ileostomy is connected to a collecting bag for collecting the intestinal fluid from an interior of the ileum. And then an opening of the abdomen of the patient is sutured.

Laparotomy will cause a larger wound and the surrounding portion of the ileum (around the active opening of the ileum) is exposed to an exterior of the abdominal wall; hence, the patient is more susceptible to infection. The surrounding portion of the ileum (around the active opening of the ileum) is subject to greater tension because it must be stretch to be secured to maintain stability; in addition, since there are no other tools to assist to secure the ileum inside the abdomen of the patient, the surrounding portion of the ileum (around the active opening of the ileum) and a portion of the ileum within the incision of the abdominal wall are more likely to be pulled and stressed due to the patient's movements; hence, necrosis may occur, and it is easy to cause early complications (within 30 days, such as inappropriate location, peristomal skin complications and stoma retraction) or late complications (after 30 days, such as parastomal hernia, stoma stenosis, stoma prolapse and parastomal infection). Moreover, if too many blood vessels are cut off in the surrounding portion of the ileum (around the active opening of the ileum) or if the blood vessels are blocked during suturing, necrosis may occur too. Furthermore, the connection between the ileostomy and the collecting bag likely cause leakage of the intestinal fluid.

### SUMMARY OF THE INVENTION

The main technical problem that the present invention is seeking to solve is how to provide an artificial intestinal device and a surgical method using the artificial intestinal device to reduce the risk of infection, strengthen the fixation of the ileum in the patient's abdomen, reduce the risk of ileostomy necrosis, early complications and late complications, and prevent leakage of the intestinal fluid.

In order to solve the above described problems and to achieve the expected effect, the present invention provides a surgical method using an artificial intestinal device, wherein the artificial intestinal device includes an artificial ileal tube, a guiding member and a fixing member, wherein the artificial ileal tube is flexible, the artificial ileal tube includes a middle tube portion and an inflated collector portion, the inflated collector portion is disposed at one end of the middle tube portion, the inflated collector portion is in communication with the middle tube portion, a width of the inflated collector portion of the artificial ileal tube is greater than a diameter of the middle tube portion of the artificial ileal tube and a diameter of the guiding member, wherein one end of the guiding member has a tip portion, the other end of the guiding member is detachably connected to the other end of the middle tube portion of the artificial ileal tube to form an assembly, wherein the fixing member is flexible, the fixing member includes a first portion, a second portion and a cushion portion between the first portion and the second portion, the surgical method comprising following steps of: Step A: making a first incision of an abdominal wall of a patient, wherein the first incision is around a right lower corner of an abdomen of the patient; Step B: making a first incision of an ileum in a near terminal ileum portion of the ileum, wherein a size of the first incision of the ileum is approximately equal to or greater than the width of the inflated collector portion of the artificial ileal tube; Step C: passing the guiding member of the assembly into an interior of the ileum through the first incision of the ileum; Step D: making a second incision of the ileum in the near terminal ileum portion of the ileum such that the tip portion of the guiding member is capable of being observed from an exterior of the ileum through the second incision of the ileum, wherein the first incision of the ileum is between an terminal ileum and the second incision of the ileum, wherein a size of the second incision of the ileum is smaller than the width of the inflated collector portion of the artificial ileal tube, the size of the second incision of the ileum is approximately equal to or a little bit smaller than the diameter of the middle tube portion of the artificial ileal tube; Step E: pulling the tip portion of the guiding member out of the ileum from the exterior of the ileum through the second incision of the ileum, such that the guiding member of the assembly is pulled out of the ileum through the second incision of the ileum and the artificial ileal tube of the assembly is passed into the interior of the ileum through the first incision of the ileum, and continuing pulling the guiding member until the middle tube portion of the artificial ileal tube is pulled out of the ileum through the second incision of the ileum and the inflated collector portion of the artificial ileal tube is stuck at the second incision of the ileum, such that the inflated collector portion of the artificial ileal tube is stayed in the interior of the ileum; Step F: suturing the first incision of the ileum; Step G: passing one of the first portion and the second portion of the fixing member through a mesentery behind the near terminal ileum portion of the ileum, such that the cushion portion of the fixing member is suspended beneath the near terminal ileum portion of the ileum; Step H: making a for-tube-incision and a for-fixing-incision of the abdominal wall; Step I: passing the tip portion of the guiding member out of the abdominal wall through the for-tube-incision of the abdominal wall and passing one end of the first portion and one end of the second portion of the fixing member out of the abdominal wall through the for-fixing-incision of the abdominal wall respectively; Step J: pulling the one end of the first portion and the one end of the second portion of the fixing member and the tip portion of the guiding member outward from an exterior of the abdominal wall, such that the first portion and the second portion of the fixing member, the guiding member and the middle tube portion of the artificial ileal tube are exposed to the exterior of the abdominal wall, and the cushion portion of the fixing member gradually pushes the near terminal ileum portion of the ileum closer to the abdominal wall, and continuing pulling the first portion and the second portion of the fixing member and the middle tube portion of the artificial ileal tube outward from the exterior of the abdominal wall, such that the second incision of the ileum is adjacent to or attached to an inner surface of the abdominal wall; Step K: fixing the fixing member from the exterior of the abdominal wall, such that the near terminal ileum portion of the ileum is fixed by the cushion portion of the fixing member; and Step L: suturing the first incision of the abdominal wall.

In implementation, before the Step B, the surgical method further comprises a following step of: making a second incision of the abdominal wall.

In implementation, the Step B, the Step C, the Step D, the Step E, the Step F and the Step G are performed in an abdomen cavity of the patient.

In implementation, after the Step A and before the Step C, the surgical method further comprises a following step of: passing the assembly into an abdomen cavity of the patient through one of the first incision of the abdominal wall and the second incision of the abdominal wall.

In implementation, after the Step A and before the Step G, the surgical method further comprises a following step of: passing the fixing member into an abdomen cavity of the patient through one of the first incision of the abdominal wall and the second incision of the abdominal wall.

In implementation, after the Step J, the surgical method further comprises a following step of: suturing the second incision of the abdominal wall.

In implementation, after the Step A and before the Step B, the surgical method further comprises a following step of: pulling the near terminal ileum portion of the ileum out of the abdominal wall from an abdomen cavity of the patient through the first incision of the abdominal wall.

In implementation, after the Step G and before the Step I, the surgical method further comprises a following step of: passing the near terminal ileum portion of the ileum, the fixing member and the assembly into the abdomen cavity of the patient through the first incision of the abdominal wall.

In implementation, after the Step J, the surgical method further comprises following steps of: detaching the guiding member from the middle tube portion of the artificial ileal tube; and connecting a collecting device to the middle tube portion of the artificial ileal tube.

In implementation, the artificial intestinal device further includes a fixing flange, after the Step J, the surgical method further comprises a following step of: Step M: sleeving the fixing flange on the middle tube portion of the artificial ileal tube and moving the fixing flange to attach to an outer surface of the abdominal wall, such that the middle tube portion of the artificial ileal tube passes through a through hole of the fixing flange and the middle tube portion of the artificial ileal tube is fixed by the through hole of the fixing flange.

In implementation, after the Step J, the surgical method further comprises following steps of: Step N: detaching the guiding member from the middle tube portion of the artificial ileal tube; and Step O: connecting a collecting device to the middle tube portion of the artificial ileal tube; wherein the Step N is performed after the Step M and before the Step O or the Step M is performed after the Step N and before the Step O.

In implementation, after the middle tube portion of the artificial ileal tube is passed through the through hole of the fixing flange, the surgical method further comprises a following step of: passing a suture through two through holes of the fixing flange and the abdominal wall respectively for fixing the fixing flange to the outer surface of the abdominal wall by the suture.

In implementation, the artificial intestinal device further includes a fixing plate, wherein, in the Step K, the first portion and the second portion of the fixing member are passed through two elongated notches of the fixing plate toward two through holes of the fixing plate respectively and the fixing plate is moved to attach to an outer surface of the abdominal wall, such that the first portion and the second portion of the fixing member are fixed in the two through holes of the fixing plate.

In implementation, the Step G is performed after the Step A and before the Step H.

Furthermore, the present invention also provides an artificial intestinal device, which comprises a fixing member, an artificial ileal tube and a guiding member. The fixing member includes a first portion, a second portion and a cushion portion between the first portion and the second portion. The fixing member is flexible. The first portion and the second portion have a long stripe shape. The cushion portion is used for being suspended beneath a near terminal ileum portion of an ileum after one of the first portion and the second portion is passed through a mesentery behind the near terminal ileum portion of the ileum. The artificial ileal tube includes a middle tube portion and an inflated collector portion. The artificial ileal tube is flexible. The inflated collector portion is disposed at one end of the middle tube portion. The inflated collector portion is in communication with the middle tube portion. The middle tube portion includes a connection tube portion at the other end of the middle tube portion. A width of the inflated collector portion is greater than a diameter of the middle tube portion. The inflated collector portion is used for collecting an intestinal fluid from an interior of the ileum. The guiding member has a tip portion at one end and a connection portion at the other end. The connection portion of the guiding member is detachably connected to the connection tube portion of the middle tube portion of the artificial ileal tube to form an assembly. The width of the inflated collector portion of the artificial ileal tube is greater than a diameter of the guiding member. The tip portion of the guiding member is used for guiding the assembly passing into the interior of the ileum through a first incision of the ileum in the near terminal ileum portion of the ileum and then guiding the assembly except the inflated collector portion of the artificial ileal tube passing out of the ileum through a second incision of the ileum in the near terminal ileum portion of the ileum such that the inflated collector portion of the artificial ileal tube is stuck at the second incision of the ileum and such that the inflated collector portion of the artificial ileal tube is stayed in the interior of the ileum, and then guiding the tip portion of the guiding member passing out of an abdominal wall through a for-tube-incision of the abdominal wall, wherein after one end of the first portion and one end of the second portion of the fixing member are passed out of the abdominal wall through a for-fixing-incision of the abdominal wall, and the tip portion of the guiding member and the first portion and the second portion of the fixing member are pulled outward from an exterior of the abdominal wall, such that the assembly except the inflated collector portion of the artificial ileal tube are exposed to the exterior of the abdominal wall and such that the cushion portion of the fixing member gradually pushes the near terminal ileum portion of the ileum closer to the abdominal wall and the second incision of the ileum is adjacent to or attached to an inner surface of the abdominal wall, and then the fixing member is fixed from the exterior of the abdominal wall, such that the near terminal ileum portion of the ileum is fixed by the cushion portion of the fixing member, wherein the first incision of the ileum is between a terminal ileum and the second incision of the ileum. A size of the first incision of the ileum is approximately equal to or greater than the width of the inflated collector portion of the artificial ileal tube. A size of the second incision of the ileum is smaller than the width of the inflated collector portion of the artificial ileal tube. The size of the second incision of the ileum is approximately equal to or a little bit smaller than the diameter of the middle tube portion of the artificial ileal tube. After the connection portion of the guiding member is detached from the connection tube portion of the middle tube portion of the artificial ileal tube, the intestinal fluid collected by the inflated collector portion of the artificial ileal tube is guided to flow from the interior of the ileum to the exterior of the abdominal wall through the middle tube portion of the artificial ileal tube.

In implementation, the artificial intestinal device further comprises a fixing flange, wherein the fixing flange is used for sleeving on the middle tube portion of the artificial ileal tube and then the fixing flange is moved to attach to an outer surface of the abdominal wall, such that the middle tube portion of the artificial ileal tube passes through a through hole of the fixing flange and the middle tube portion of the artificial ileal tube is fixed by the through hole of the fixing flange.

In implementation, the fixing flange further comprises a fixing structure formed on an inner surface of the through hole of the fixing flange for preventing the middle tube portion of the artificial ileal tube from slipping.

In implementation, the artificial intestinal device further comprises a fixing plate, wherein the fixing plate has two through holes and two elongated notches, one of the two elongated notches extends from one of the two through holes to a periphery of the fixing plate along one direction from the one of the two through holes toward the periphery of the fixing plate, the other one of the two elongated notches extends from the other one of the two through holes to the periphery of the fixing plate along an inverse direction of the one direction, wherein a diameter of each of the two elongated notches is smaller than a diameter of each of the two through holes, the diameter of each of the two through holes is smaller than a diameter of the first portion and a diameter of the second portion of the fixing member, wherein after the first portion and the second portion of the fixing member are passed through the two elongated notches of the fixing plate toward the two through holes of the fixing plate respectively and the fixing plate is moved to attach to an outer surface of the abdominal wall, such that the first portion and the second portion of the fixing member are fixed in the two through holes of the fixing plate so as to fix the fixing member from the exterior of the abdominal wall.

In implementation, the one end of the second portion of the fixing member further includes a through hole, wherein after the first portion and the second portion of the fixing member are fixed in the two through holes of the fixing plate, the one end of the first portion of the fixing member is passed through the through hole of the one end of the second portion of the fixing member, such that the fixing member is fixed more tightly.

In implementation, the cushion portion of the fixing member includes a plurality of sub-portions, wherein a diameter of a junction between any two adjacent sub-portions of the plurality of sub-portions is smaller than a diameter of each of the two adjacent sub-portions.

In implementation, the artificial intestinal device further comprises a obturator cap, wherein after the artificial ileal tube is pulled out of the ileum through the second incision of the ileum and out of an abdomen cavity through the for-tube-incision of the abdominal wall, a protrusion portion of the obturator cap is capable of being passed through the for-tube-incision of the abdominal wall and plugged into the second incision of the ileum, wherein the obturator cap is made of biodegradable materials for assisting the second incision of the ileum and the for-tube-incision of the abdominal wall healing.

For further understanding the characteristics and effects of the present invention, some preferred embodiments referred to drawings are in detail described as follows.

### BRIEF DESCRIPTION OF DRAWINGS

FIG. 1 is a schematic perspective view of an embodiment of an artificial ileal tube of an artificial intestinal device of the present invention.

FIG. 2 is a schematic perspective view of an embodiment of a guiding member of an artificial intestinal device of the present invention.

FIG. 3 is a schematic perspective view of an assembly of the artificial ileal tube of FIG. 1 and the guiding member of FIG. 2.

FIG. 4 is a schematic perspective view of an embodiment of a fixing member of an artificial intestinal device of the present invention.

FIG. 5 is a schematic top view of an embodiment of a fixing plate of an artificial intestinal device of the present invention.

FIG. 6 is a schematic cross-sectional view of the fixing plate of FIG. 5.

FIG. 7 is a schematic top view of an embodiment of a fixing flange of an artificial intestinal device of the present invention.

FIG. 8 is a schematic cross-sectional view of the fixing flange of FIG. 7.

FIG. 9 is a schematic top view of an embodiment of an obturator cap of an artificial intestinal device of the present invention.

FIG. 10 is a schematic cross-sectional view of the obturator cap of FIG. 9.

FIG. 11 is a schematic front view of an exterior of an abdomen wall of a patient undergoing surgery.

FIG. 12-15 are respectively schematic front views of an abdomen cavity of the patient undergoing surgery.

FIG. 16 is a schematic cross-sectional view of the abdomen cavity of the patient undergoing surgery.

FIG. 17 is a schematic front view of the exterior of the abdomen wall of the patient undergoing surgery.

FIG. 18 is a schematic perspective view of another embodiment of an artificial ileal tube and a guiding member of an artificial intestinal device of the present invention.

FIG. 19 is a schematic perspective view of another embodiment of a fixing member of an artificial intestinal device of the present invention.

FIG. 20 is a schematic cross-sectional view of another embodiment of a fixing flange of an artificial intestinal device of the present invention.

FIG. 21-26 are schematic front views of an exterior of an abdomen wall of a patient undergoing surgery.

### DETAILED DESCRIPTIONS OF PREFERRED EMBODIMENTS

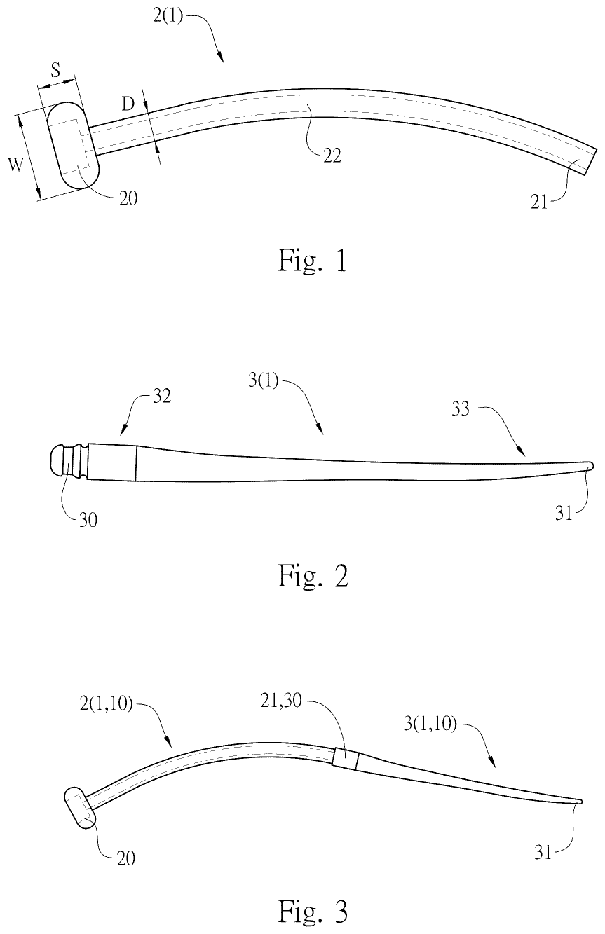

Please refer to FIGS. 1-2 and 4, which respectively show schematic perspective views of an embodiment of an artificial ileal tube, a guiding member and a fixing member of an artificial intestinal device of the present invention. Please also refer to FIG. 3, which shows a schematic perspective view of an assembly of the artificial ileal tube of FIG. 1 and the guiding member of FIG. 2. An artificial intestinal device 1 of the present invention comprises an artificial ileal tube 2, a guiding member 3 and a fixing member 4.

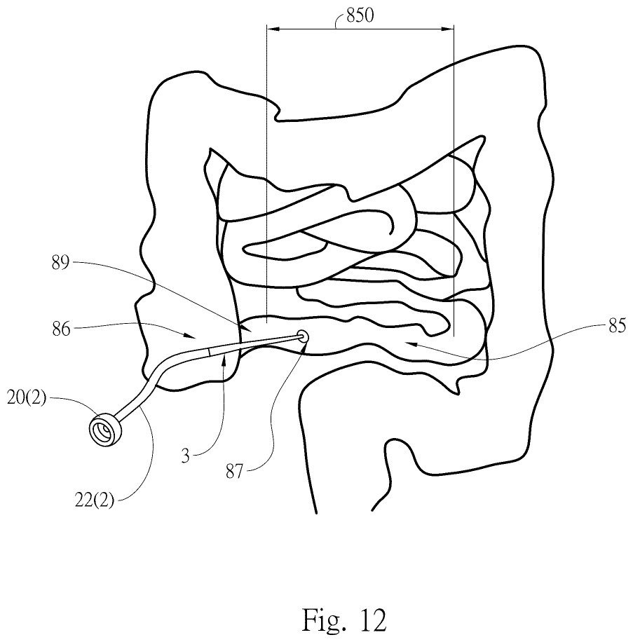

The artificial ileal tube 2 comprises an inflated collector portion 20 and a middle tube portion 22. The inflated collector portion 20 is disposed at one end of the middle tube portion 22. The inflated collector portion 20 is in communication with the middle tube portion 22. The middle tube portion 22 includes a connection tube portion 21 at the other end of the middle tube portion 22. A diameter D of the middle tube portion 22 is smaller than a width W of the inflated collector portion 20. The artificial ileal tube 2 (including the inflated collector portion 20) is elastic and flexible. The artificial ileal tube 2 (including the inflated collector portion 20) may be made of silicone, PU, PE of rubber. The artificial ileal tube 2 has two openings. One of the two openings is located at the connection tube portion 21 of the middle tube portion 22. The other one of the two openings is located at the inflated collector portion 20. The two openings are in communication with each other through the connection tube portion 21 of the middle tube portion 22 and the inflated collector portion 20. In current embodiment, the width W of the inflated collector portion 20 is greater than or a little bit greater than an inner size or an inner diameter of an ileum 85 (please refer to FIG. 12), especially greater than an inner size or an inner diameter of a near terminal ileum portion 850 of the ileum 85, such that the ileum 85 (especially the near terminal ileum portion 850 of the ileum 85) is slightly expanded by the elastic and flexible inflated collector portion 20. The diameter D of the middle tube portion 22 is smaller than the inner size or the inner diameter of the ileum 85 (especially the inner size or the inner diameter of the near terminal ileum portion 850 of the ileum 85). In some other embodiments, the width W of the inflated collector portion 20 is approximately equal to the inner size or the inner diameter of the ileum 85 (especially the inner size or the inner diameter of the near terminal ileum portion 850 of the ileum 85). In some other embodiments, the width W of the inflated collector portion 20 is no less than the inner size or the inner diameter of the ileum 85 (especially the inner size or the inner diameter of the near terminal ileum portion 850 of the ileum 85).

The guiding member 3 tapers gradually from a first end 32 to a second end 33; i.e., a diameter of the first end 32 of the guiding member 3 is greater than a diameter of the second end 33 of the guiding member 3. The first end 32 of the guiding member 3 has a connection portion 30. The diameter of the first end 32 of the guiding member 3 is smaller than the width W of the inflated collector portion 20 of the artificial ileal tube 2. The diameter of the first end 32 of the guiding member 3 may be about the same as the diameter D of the middle tube portion 22 of the artificial ileal tube 2. The second end 33 of the guiding member 3 has a tip portion 31 (for example, a pointed bit tip portion). In FIG. 3, the connection portion 30 of the guiding member 3 is detachably connected to the connection tube portion 21 of the middle tube portion 22 of the artificial ileal tube 2 to form an assembly 10. The guiding member 3 may be made of silicone, PU, PE, rubber, plastic, stainless or ceramic. The diameter of the first end 32 of the guiding member 3 is smaller than the inner size or the inner diameter of an ileum 85, especially smaller than the inner size or the inner diameter of a near terminal ileum portion 850 of the ileum 85.

The fixing member 4 comprises a first portion 40, a cushion portion 42 and a second portion 41. A first end 45 of the cushion portion 42 is connected to a second end 47 of the first portion 40. A second end 46 of the cushion portion 42 is connected to a first end 48 of the second portion 41. The cushion portion 42 is an enlarged portion. A diameter of the cushion portion 42 between the first end 45 and the second end 46 of the cushion portion 42 is greater than a diameter of the first portion 40 and also greater than a diameter of the second portion 41. The fixing member 4 (including the cushion portion 42) is elastic and flexible. The fixing member 4 (including the cushion portion 42) may be made of silicone, PU, PE of rubber. In current embodiment, the fixing member 4 is a fixing tube. The first portion 40 is in communication with the cushion portion 42. The cushion portion 42 is in communication with the second portion 41. The fixing member 4 has an opening at a first end 43 of the first portion 40 and an opening at a second end 44 of the second portion 41. The opening at the first end 43 of the first portion 40 is in communication with the opening at the second end 44 of the second portion 41 through the first portion 40, the cushion portion 42 and the second portion 41.

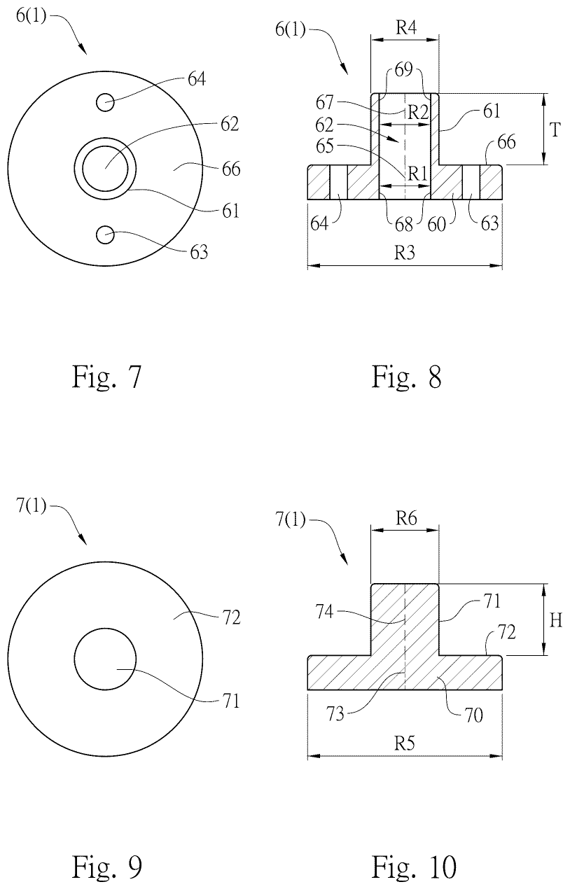

Please refer to FIGS. 5, 7 and 9, which respectively show schematic top views of an embodiment of a fixing plate, a fixing flange and an obturator cap of an artificial intestinal device of the present invention. Please also refer to FIGS. 6, 8 and 10, which respectively show schematic cross-sectional views of the fixing plate of FIG. 5, the fixing flange of FIG. 7 and the obturator cap of FIG. 9. The artificial intestinal device 1 of the present invention further comprises a fixing plate 5, a fixing flange 6 and an obturator cap 7.

In current embodiment, the fixing plate 5 is a circular fixing plate. The fixing plate 5 has a first through hole 50, a second through hole 51, a first elongated notch 52 and a second elongated notch 53. The first through hole 50 and the second through hole 51 are respectively located on two opposite sides of a center of the fixing plate 5. The first through hole 50 and the second through hole 51 penetrate the fixing plate 5 respectively. The first elongated notch 52 extends from the first through hole 50 to a periphery of the fixing plate 5 along a first radially outward direction 55; while the second elongated notch 53 extends from the second through hole 51 to the periphery of the fixing plate 5 along a second radially outward direction 56, wherein the second radially outward direction 56 is opposite to the first radially outward direction 55. A width of the first elongated notch 52 is a little bit smaller than a diameter of the first through hole 50. A width of the second elongated notch 53 is a little bit smaller than a diameter of the second through hole 51. The diameter of the first through hole 50 and the diameter of the second through hole 51 are smaller than the diameter of the first portion 40 and the diameter of the second portion 41 respectively. The fixing plate 5 may be made of silicone, PU, PE of rubber.

The fixing flange 6 comprises a plate 60 and a protrusion portion 61. In current embodiment, the plate 60 is a cylindrical annulus plate; the protrusion portion 61 is a cylindrical annulus protrusion portion. An inner diameter R1 of the plate 60 is equal to an inner diameter R2 of the protrusion portion 61. The protrusion portion 61 is protruded from a top surface 66 of the plate 60 and an axis 65 of the plate 60 is coincident with an axis 67 of the protrusion portion 61. An outer diameter R3 of the plate 60 is greater than an outer diameter R4 of the protrusion portion 61. The plate 60 has two through holes 63, 64 beside the protrusion portion 61. The through holes 63, 64 are located on two opposite sides of the plate 60. Through holes 63, 64 penetrate the plate 60 respectively. A through hole 62 (in current embodiment, the through hole 62 is a cylindrical through hole) is defined by an inner surface 68 of the plate 60 and an inner surface 69 of the protrusion portion 61; i.e., the through hole 62 penetrates the fixing flange 6 (penetrates the plate 60 and the protrusion portion 61), and a diameter of the through hole 62 is equal to the inner diameter R1 of the plate 60 and also equal to the inner diameter R2 of the protrusion portion 61. The fixing flange 6 may be made of silicone, PU, PE of rubber.

The obturator cap 7 comprises a plate 70 and a protrusion portion 71. In current embodiment, the plate 70 is a circular plate; the protrusion portion 71 is a cylindrical protrusion portion. The protrusion portion 71 is protruded from a top surface 72 of the plate 70 and an axis 73 of the plate 70 is coincident with an axis 74 of the protrusion portion 71. An outer diameter R5 of the plate 70 is greater than an outer diameter R6 of the protrusion portion 71. In some embodiments, the obturator cap 7 may be made of biodegradable materials, such as, hyaluronic acid, polysaccharide, collagen, hydrogen gel etc. The obturator cap 7 made of biodegradable materials may assist the incision healing. In some embodiments, as the obturator cap 7 is biodegraded, the incision is healed. In some other embodiments, the plate 70 may be not a circular shape.

### First Embodiment

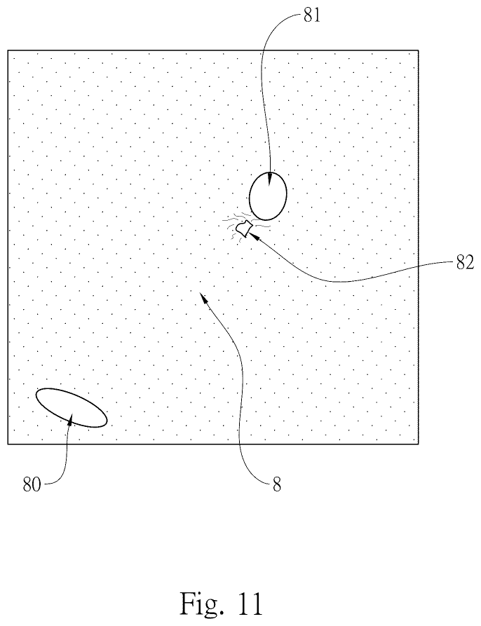

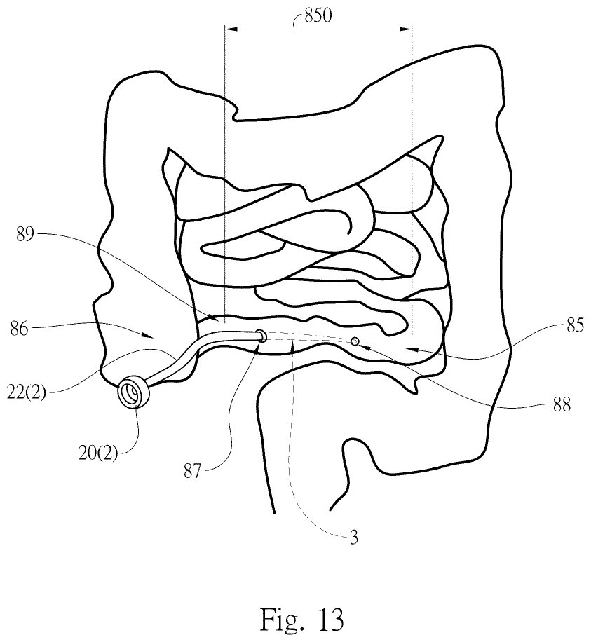

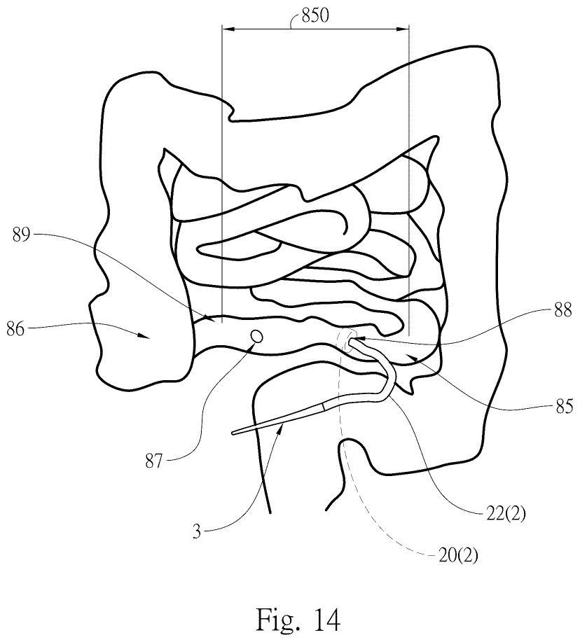

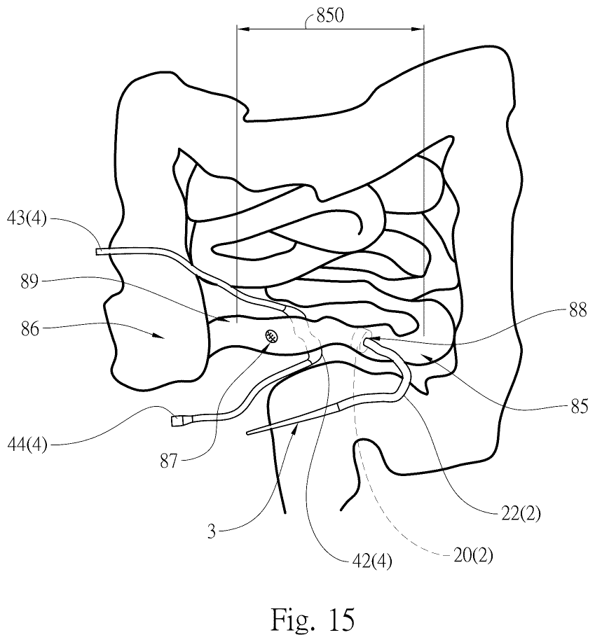

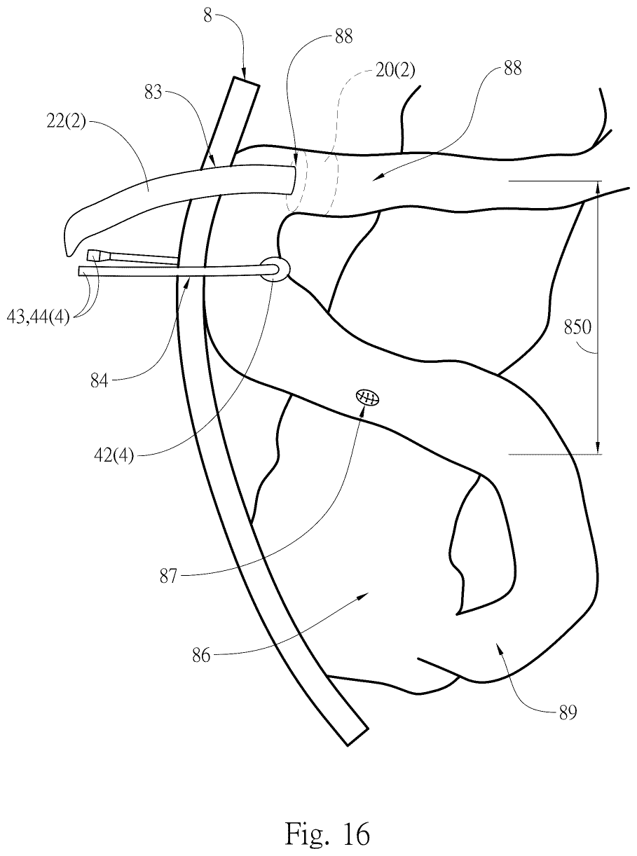

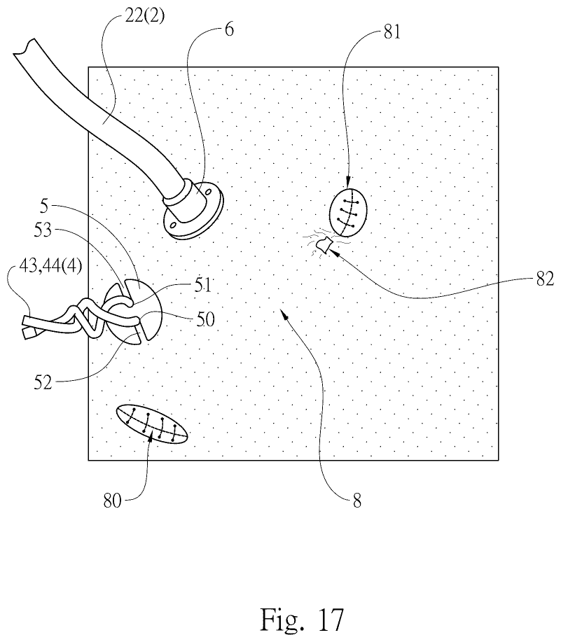

The present invention also provides a surgical method using the artificial intestinal device 1 of the present invention. The artificial intestinal device 1 of the present invention comprises an artificial ileal tube 2 (elastic and flexible), a guiding member 3, a fixing member 4 (elastic and flexible), a fixing plate 5 and a fixing flange 6. The surgical method using the artificial intestinal device 1 of the present invention may be a minimally invasive surgery. The surgical method using the artificial intestinal device 1 of the present invention comprises following steps of: Step A1: (please refer to FIG. 11, which shows a schematic front view of an exterior of an abdomen wall of a patient undergoing surgery) making a first incision 80 and a second incision 81 of an abdominal wall 8 of a patient, wherein the first incision 80 of the abdominal wall 8 is around a right lower corner of an abdomen of the patient (as shown in a left lower corner of FIG. 11); while the second incision 81 of the abdominal wall 8 is near a belly button 82 of the patient (in some other embodiments, the second incision 81 of the abdominal wall 8 is located to the left of the belly button 82 or the upper left of the belly button 82), wherein the second incision 81 of the abdominal wall 8 is used for passing some surgical tools of minimally invasive surgery into an abdomen cavity of the patient through the second incision 81 of the abdominal wall 8; while the first incision 80 of the abdominal wall 8 is used for passing other the surgical tools of minimally invasive surgery into the abdomen cavity of the patient through the first incision 80 of the abdominal wall 8; wherein the artificial intestinal device 1 of the present invention (such as the assembly 10 (including the artificial ileal tube 2 and the guiding member 3) and the fixing member 4) can be passed into the abdomen cavity of the patient through the first incision 80 of the abdominal wall 8 or through the second incision 81 of the abdominal wall 8; Step B1: (please refer to FIG. 12, which shows a schematic front view of an abdomen cavity of the patient undergoing surgery) identifying a location of a terminal ileum 89 of an ileum 85 in the abdomen cavity of the patient (the terminal ileum 89 of the ileum 85 is where the ileum 85 connected to a cecum 86); Step C1: passing the assembly 10 (including the artificial ileal tube 2 and the guiding member 3) into the abdomen cavity of the patient through the first incision 80 of the abdominal wall 8 or through the second incision 81 of the abdominal wall 8, wherein a length of the guiding member 3 may be about, for example, 10 cm, wherein the length of the guiding member 3 can be used as a ruler for helping a surgeon in judging distance or length in the abdomen cavity of the patient; Step D1: making a first incision 87 of the ileum 85 in a near terminal ileum portion 850 of the ileum 85, wherein the first incision 87 of the ileum 85 may be about, for example, a first distance away from the terminal ileum 89 of the ileum 85, wherein the first distance may be about the length of the guiding member 3 (for example, about 10 cm), wherein a size or an area of the first incision 87 of the ileum 85 may be approximately equal to or greater than a width W of an inflated collector portion 20 of the artificial ileal tube 2 of the assembly 10, wherein the width W of the inflated collector portion 20 of the artificial ileal tube 2 of the assembly 10 is no less than, greater than, a little bit greater than or approximately equal to an inner size of the near terminal ileum portion 850 of the ileum 85; Step E1: passing the guiding member 3 of the assembly 10 (starting from a tip portion 31 of the guiding member 3) into an interior of the ileum 85 through the first incision 87 of the ileum 85 in a direction away from the terminal ileum 89, and stop passing the guiding member 3 of the assembly 10 when the guiding member 3 of the assembly 10 is totally inserted into the interior of the ileum 85 through the first incision 87 of the ileum 85 (please refer to FIG. 13, which shows a schematic front view of the abdomen cavity of the patient undergoing surgery); i.e., the assembly 10 is inserted into the interior of the ileum 85 for about the length of the guiding member 3 (for example, about 10 cm); Step F1: making a second incision 88 of the ileum 85 in the near terminal ileum portion 850 of the ileum 85, wherein the first incision 87 of the ileum 85 is between the second incision 88 of the ileum 85 and the terminal ileum 89, wherein the second incision 88 of the ileum 85 is about, for example, a second distance away from the first incision 87 of the ileum 85, wherein the second distance may be about the length of the guiding member 3 (for example, about 10 cm); and when the guiding member 3 of the assembly 10 is totally inserted into the interior of the ileum 85 through the first incision 87 of the ileum 85, the second incision 88 of the ileum 85 is about at the location of the tip portion 31 of guiding member 3 such that the tip portion 31 of guiding member 3 is exposed to an exterior of the ileum 85 within the abdomen cavity through the second incision 88 of the ileum 85; i.e., the tip portion 31 of guiding member 3 can be observed (for example, by laparoscopic endoscopy) through the second incision 88 of the ileum 85 from the location in the abdomen cavity but in the exterior of the ileum 85, wherein a size or an area of the second incision 88 of the ileum 85 is approximately equal to or a little bit smaller than the diameter D of the middle tube portion 22 of the artificial ileal tube 2, wherein the size or the area of the second incision 88 of the ileum 85 is smaller than the size or the area of the first incision 87 of the ileum 85 and smaller than the width W of the inflated collector portion 20 of the artificial ileal tube 2; Step G1: (please refer to FIG. 14, which shows a schematic front view of the abdomen cavity of the patient undergoing surgery) pulling the tip portion 31 of the guiding member 3 of the assembly 10 from the exterior of the ileum 85 through the second incision 88 of the ileum 85 such that the guiding member 3 of the assembly 10 is pulled out of the ileum 85 (remains in the abdomen cavity) through the second incision 88 of the ileum 85, and, in the meantime, the middle tube portion 22 and the inflated collector portion 20 of the artificial ileal tube 2 of the assembly 10 are passed into the interior of the ileum 85 through the first incision 87 of the ileum 85; and continuing pulling the guiding member 3 of the assembly 10 until the middle tube portion 22 of the artificial ileal tube 2 of the assembly 10 is pulled out of the ileum 85 (remains in the abdomen cavity) through the second incision 88 of the ileum 85 and the inflated collector portion 20 of the artificial ileal tube 2 is stuck in the second incision 88 of the ileum 85, such that the inflated collector portion 20 of the artificial ileal tube 2 is stayed in the interior of the ileum 85, wherein an opening of the inflated collector portion 20 of the artificial ileal tube 2 is facing to a direction away from the terminal ileum 89; Step H1: (please refer to FIG. 15, which shows a schematic front view of the abdomen cavity of the patient undergoing surgery) suturing the first incision 87 of the ileum 85; Step I1: passing the fixing member 4 into the abdomen cavity through the first incision 80 of the abdominal wall 8 or through the second incision 81 of the abdominal wall 8; Step J1: passing one of a first end 43 of a first portion 40 and a second end 44 of a second portion 41 of the fixing member 4 through a mesentery behind the near terminal ileum portion 850 of the ileum 85 such that a cushion portion 42 of the fixing member 4 is suspended beneath the near terminal ileum portion 850 of the ileum 85; Step K1: (please refer to FIG. 16, which shows a schematic cross-sectional view of the abdomen cavity of the patient undergoing surgery) making a third incision 83 of the abdominal wall 8 (the third incision 83 is also denoted as a for-tube-incision) and a fourth incision 84 of the abdominal wall 8 (the fourth incision 84 is also denoted as a for-fixing-incision), wherein a location of the fourth incision 84 of the abdominal wall 8 is lower than a location of the third incision 83 of the abdominal wall 8 but higher than a location of the first incision 80 of the abdominal wall 8; Step L1: passing the first end 43 of the first portion 40 and the second end 44 of the second portion 41 of the fixing member 4 out of the abdominal wall 8 through the fourth incision 84 of the abdominal wall 8; Step M1: passing the tip portion 31 of the guiding member 3 of the assembly 10 out of the abdominal wall 8 through the third incision 83 of the abdominal wall 8; Step N1: pulling the first end 43 of the first portion 40 and the second end 44 of the second portion 41 of the fixing member 4 outward from an exterior of the abdominal wall 8, and, in the meantime, pulling the tip portion 31 of the guiding member 3 of the assembly 10 outward from the exterior of the abdominal wall 8, such that the first portion 40 and the second portion 41 of the fixing member 4, the guiding member 3 and the middle tube portion 22 of the artificial ileal tube 2 are exposed to the exterior of the abdominal wall 8, and the cushion portion 42 of the fixing member 4 gradually pushes the near terminal ileum portion 850 of the ileum 85 closer to the abdominal wall 8, and continuing pulling the first portion 40 and the second portion 41 of the fixing member 4 and pulling the middle tube portion 22 of the artificial ileal tube 2 outward from the exterior of the abdominal wall 8, such that the second incision 88 of the ileum 85 is adjacent to or attached to an inner surface of the abdominal wall 8, wherein since the cushion portion 42 of the fixing member 4 is an enlarged portion (especially having a larger cross-section) and is elastic and flexible, the near terminal ileum portion 850 of the ileum 85 pushed by the enlarged cushion portion 42 of the fixing member 4 will not be hurt by local tightening; Step O1: (please refer to FIG. 17, which shows a schematic front view of the exterior of the abdomen wall of the patient undergoing surgery) passing the first portion 40 of the fixing member 4 through a first elongated notch 52 of a fixing plate 5 from a periphery of the fixing plate 5 to a first through hole 50 of the fixing plate 5 along an inverse direction of a first radially outward direction 55 and passing the second portion 41 of the fixing member 4 through a second elongated notch 53 of the fixing plate 5 from the periphery of the fixing plate 5 to a second through hole 51 of the fixing plate 5 along an inverse direction of a second radially outward direction 56, and moving the fixing plate 5 to attach to an outer surface of the abdominal wall 8, such that the first portion 40 and the second portion 41 of the fixing member 4 are fixed in the first through hole 50 and the second through hole 51 of the fixing plate 5 respectively (since a diameter of the first through hole 50 and a diameter of the second through hole 51 are smaller than a diameter of the first portion 40 and a diameter of the second portion 41 respectively; wherein the fixing member 4 is fixed from the exterior of the abdominal wall 8) and the near terminal ileum portion 850 of the ileum 85 is fixed by the cushion portion 42 of the fixing member 4, wherein the fixing plate 5 can prevent the fixing member 4 (the cushion portion 42) and the second incision 88 of the ileum 85 from slipping, shifting or moving, wherein the first portion 40 and the second portion 41 of the fixing member 4 can further be tied to secure the fixing member 4; Step P1: sleeving a fixing flange 6 on the middle tube portion 22 of the artificial ileal tube 2 and moving the fixing flange 6 to attach to the outer surface of the abdominal wall 8, such that the middle tube portion 22 of the artificial ileal tube 2 passes through a through hole 62 of the fixing flange 6 and the middle tube portion 22 of the artificial ileal tube 2 is sleeved and fixed by the through hole 62 of the fixing flange 6, wherein the fixing flange 6 may be sleeved on the middle tube portion 22 of the artificial ileal tube 2 when the guiding member 3 is connected to the middle tube portion 22 of the artificial ileal tube 2 or the fixing flange 6 may be sleeved on the middle tube portion 22 of the artificial ileal tube 2 after the guiding member 3 is detached from the middle tube portion 22 of the artificial ileal tube 2; and then passing a suture (or sutures) through two through holes 63, 64 of the fixing flange 6 and the abdominal wall 8 respectively for fixing the fixing flange 6 to the outer surface of the abdominal wall 8 more tightly by the suture (or sutures), wherein the fixing flange 6 can prevent the inflated collector portion 20 and the middle tube portion 22 of the artificial ileal tube 2 (especially a portion of the middle tube portion 22 of the artificial ileal tube 2 in the through hole 62 of the fixing flange 6) from slipping, shifting or moving; Step Q1: removing the surgical tools of minimally invasive surgery from the abdomen cavity of the patient through the first incision 80 and/or the second incision 81 of the abdominal wall 8; and suturing the first incision 80 and the second incision 81 of the abdominal wall 8 of the patient; and Step R1: detaching the connection portion 30 of the guiding member 3 of the assembly 10 from the connection tube portion 21 of the middle tube portion 22 of the artificial ileal tube 2 of the assembly 10, wherein after the guiding member 3 is detached from the middle tube portion 22 of the artificial ileal tube 2, the connection tube portion 21 of the middle tube portion 22 of the artificial ileal tube 2 can be connected to a collecting device (not shown in Figure; for example, a collecting bag or an urinary bag), so that the inflated collector portion 20 of the artificial ileal tube 2 can collect an intestinal fluid from an interior of the ileum 85 and then the intestinal fluid collected by the inflated collector portion 20 of the artificial ileal tube 2 can be guided to flow from the interior of the ileum 85 to the exterior of the abdominal wall 8 to the collecting device (not shown in Figure) through the middle tube portion 22 of the artificial ileal tube 2. In current embodiment, the first incision 80, the second incision 81, the third incision 83 (the for-tube-incision) and the fourth incision 84 (the for-fixing-incision) of the abdominal wall 8, and the first incision 87 and the second incision 88 of the ileum 85 are relatively small. The first incision 87 of the ileum 85, the first incision 80 and the second incision 81 of the abdominal wall 8 will all be sutured. The ileum 85 will not exposed to the exterior of the abdominal wall 8. Hence, the risk of infection will be significantly reduced. The near terminal ileum portion 850 of the ileum 85 is pushed by the elastic, flexible and enlarged cushion portion 42 of the fixing member 4, so that the near terminal ileum portion 850 of the ileum 85 is fixed more tightly yet will not be hurt by local tightening; hence, the fixation of the near terminal ileum portion 850 of the ileum 85 is strengthened. There is no need to perform another surgery to remove the artificial ileal tube 2 (including the inflated collector portion 20 of the artificial ileal tube 2) and the fixing member 4 (including the cushion portion 42). Since the artificial ileal tube 2 and the fixing member 4 are elastic and flexible, the fixing member 4 is capable of being pulled out of the abdominal wall 8 through the fourth incision 84 of the abdominal wall 8 and the artificial ileal tube 2 is capable of being pulled out of the ileum 85 through the second incision 88 of the ileum 85 and being pulled out of the abdominal wall 8 through the third incision 83 of the abdominal wall 8. The second incision 88 of the ileum 85 is quite small; hence, the second incision 88 of the ileum 85 can be healed naturally and does not require sutures. The third incision 83 of the abdominal wall 8 can be sutured. The fourth incision 84 of the abdominal wall 8 can be healed naturally or can be sutured. Therefore, the risk of ileostomy necrosis is significantly reduced. The connection tube portion 21 of the middle tube portion 22 of the artificial ileal tube 2 is directly connected to the collecting device (not shown in Figure). The intestinal fluid collected by the inflated collector portion 20 of the artificial ileal tube 2 will be guided to flow from the interior of the ileum 85 to the exterior of the abdominal wall 8 to the collecting device (not shown in Figure) through the middle tube portion 22 of the artificial ileal tube 2. Hence, the leakage of the intestinal fluid will be prevented.

In some embodiments, after the Step M1 and before the Step P1, the surgical method using the artificial intestinal device 1 of the present invention may further comprises a following step of: suturing the third incision 83 of the abdominal wall 8. In some embodiments, after the Step N1 and before the Step O1, the surgical method using the artificial intestinal device 1 of the present invention may further comprises a following step of: suturing the fourth incision 84 of the abdominal wall 8. In some other embodiments, the fourth incision 84 of the abdominal wall 8 can be healed naturally and does not require sutures.

In some embodiments, after the Step B1, the near terminal ileum portion 850 of the ileum 85 is pulled out of the abdominal wall 8 through the first incision 80 of the abdominal wall 8; and then the Step C1 is skipped; and then the Step D1, the Step E1, the Step F1, the Step G1 and the Step H1 are performed in the exterior of the abdominal wall 8; and then the near terminal ileum portion 850 of the ileum 85 and the assembly 10 (including the artificial ileal tube 2 and the guiding member 3) are pushing (passing) into the abdominal wall 8 through the first incision 80 of the abdominal wall 8; and then the Step I1, the Step J1, the Step K1, the Step L1, the Step M1, the Step N1, the Step O1, the Step P1, the Step Q1 and the Step R1 are performed.

In some embodiments, the Step I1 may be performed after the Step B1 and before the Step C1, the Step D1, the Step E1, the Step F1, the Step G1, or the Step H1, wherein the Step J1 is performed after the Step I1.

In some embodiments, the Step L1 may be performed after the Step K1 and before the Step M1. In some embodiments, the Step M1 may be performed after the Step K1 and before the Step L1.

In some embodiments, the first incision 80 of the abdominal wall 8 is used for passing some surgical tools of minimally invasive surgery into the abdomen cavity of the patient through the first incision 80 of the abdominal wall 8; while the second incision 81 of the abdominal wall 8 is used for passing other the surgical tools of minimally invasive surgery and the assembly 10 (including the artificial ileal tube 2 and the guiding member 3) and the fixing member 4 of the artificial intestinal device 1 of the present invention into the abdomen cavity of the patient through the second incision 81 of the abdominal wall 8.

In some embodiments, a size or an area of the second incision 81 of the abdominal wall 8 may be smaller than a size or an area of the first incision 80 of the abdominal wall 8.

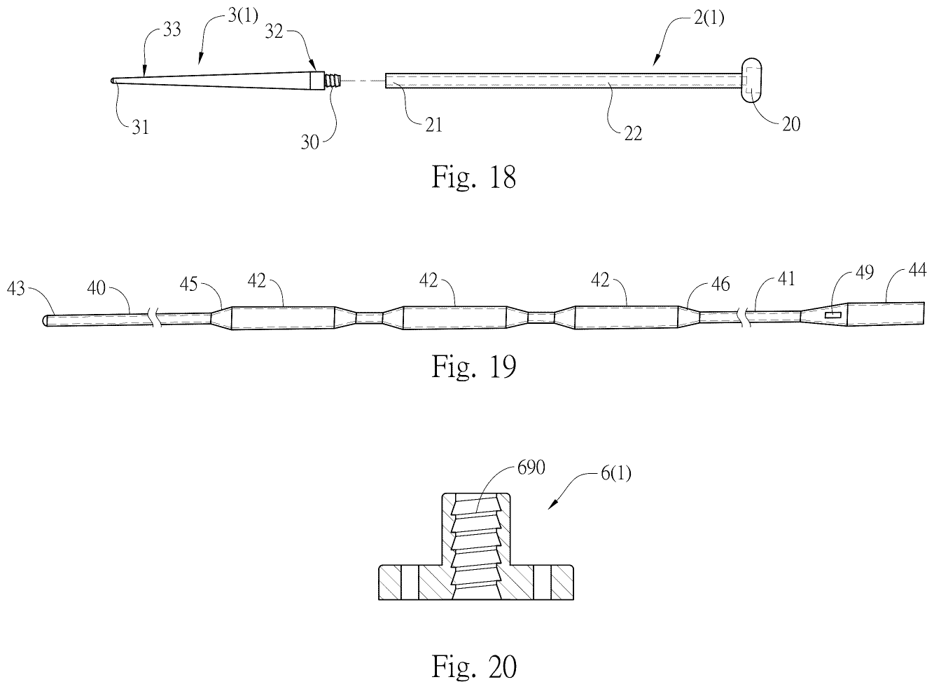

Please refer to FIG. 18, which shows a schematic perspective view of another embodiment of an artificial ileal tube and a guiding member of an artificial intestinal device of the present invention. The main structure of the embodiment of FIG. 18 is basically the same as the structure of the embodiment of FIGS. 1 and 2, except that the connection portion 30 of the guiding member 3 is capable of being screwed with the connection tube portion 21 of the middle tube portion 22 of the artificial ileal tube 2 to form the assembly 10. In current embodiment, a length of the artificial ileal tube 2 is longer than a length of the guiding member 3. In current embodiment, the guiding member 3 has a conic shape with a tip portion 31 (for example, a pointed bit tip portion). In some embodiments, a length of the artificial ileal tube 2 is about two times a length of the guiding member 3. In some other embodiments, the length of the artificial ileal tube 2 is equal to or greater than 1.5 times of the length of the guiding member 3 and equal to or smaller than two times of the length of the guiding member 3. In some embodiments, the length of the artificial ileal tube 2 is equal to or greater than two times of the length of the guiding member 3 and equal to or smaller than 2.5 times of the length of the guiding member 3. In some other embodiments, the length of the artificial ileal tube 2 is equal to or greater than 2.5 times of the length of the guiding member 3 and equal to or smaller than three times of the length of the guiding member 3. In some embodiments, the length of the artificial ileal tube 2 is equal to or greater than three times of the length of the guiding member 3 and equal to or smaller than 3.5 times of the length of the guiding member 3.

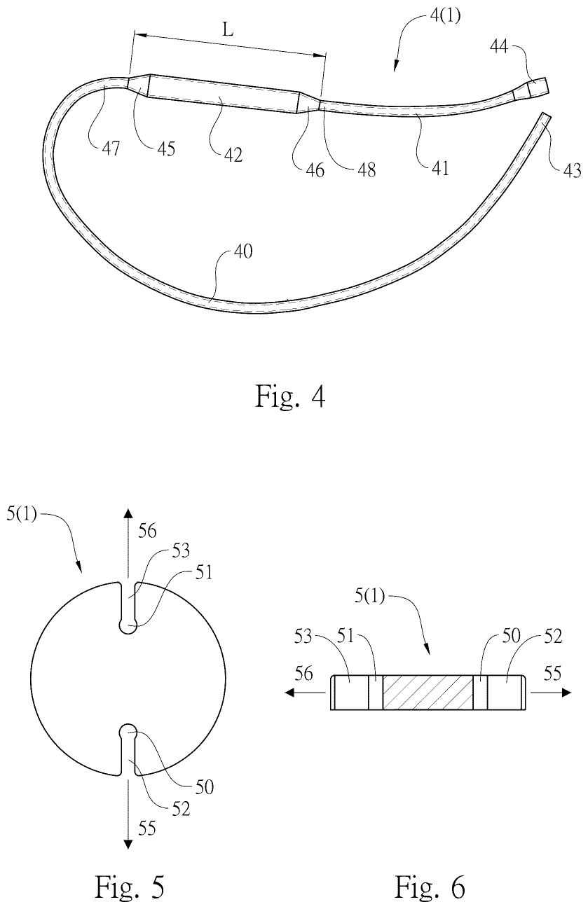

Please refer to FIG. 19, which shows a schematic perspective view of another embodiment of a fixing member of an artificial intestinal device of the present invention. The main structure of the embodiment of FIG. 19 is basically the same as the structure of the embodiment of FIG. 4, except that the cushion portion 42 of the fixing member 4 includes three sub-portions, and the second end 44 of the second portion 41 of the fixing member 4 further includes a through hole 49. A diameter of a junction between any two adjacent sub-portions is smaller than a diameter of each of the two adjacent sub-portions. In FIG. 19, a right one of the three sub-portions is adjacent to a middle one of the three sub-portions, wherein a diameter of a junction between the right one and the middle one of the three sub-portions is smaller than a diameter of the right one of the three sub-portions and a diameter of the middle one of the three sub-portions; while the middle one of the three sub-portions is adjacent to a left one of the three sub-portions, wherein a diameter of a junction between the middle one and the left one of the three sub-portions is smaller than the diameter of the middle one of the three sub-portions and a diameter of the left one of the three sub-portions. An average diameter of the cushion portion 42 of the fixing member 4 (including the three sub-portions) is greater than a diameter of the first portion 40 of the fixing member 4 and is greater than a diameter of the second portion 41 of the fixing member 4. After the first portion 40 and the second portion 41 of the fixing member 4 are fixed in the first through hole 50 and the second through hole 51 of the fixing plate 5, the first end 43 of the first portion 40 of the fixing member 4 can pass through the through hole 49 of the second portion 41 of the fixing member 4, such that the fixing member 4 is fixed more tightly. In current embodiment, the fixing member 4 is a fixing tube. The first portion 40 is in communication with the cushion portion 42 (the three sub-portions). The cushion portion 42 (the three sub-portions) is in communication with the second portion 41. The fixing member 4 has an opening at a first end 43 of the first portion 40 and an opening at a second end 44 of the second portion 41. The opening at the first end 43 of the first portion 40 is in communication with the opening at the second end 44 of the second portion 41 through the first portion 40, the cushion portion 42 (the three sub-portions) and the second portion 41.

Please refer to FIG. 20, which shows a schematic cross-sectional view of another embodiment of a fixing flange of an artificial intestinal device of the present invention. The main structure of the embodiment of FIG. 20 is basically the same as the structure of the embodiment of FIG. 8, except that the fixing flange 6 further includes a fixing structure 690 formed on the inner surface 68 and the inner surface 69 (i.e., the fixing structure 690 is formed on an inner surface of the through hole 62). The fixing structure 690 can further prevent the middle tube portion 22 of the artificial ileal tube 2 from slipping, shifting or moving. In current embodiment, the fixing structure 690 is a threaded fixing structure. In some other embodiments, the fixing structure 690 may be a wave-shaped fixing structure. In some other embodiments, the fixing structure 690 may be a jagged-shaped fixing structure. In some other embodiments, the fixing structure 690 may be an anti-slip layer.

### Second Embodiment

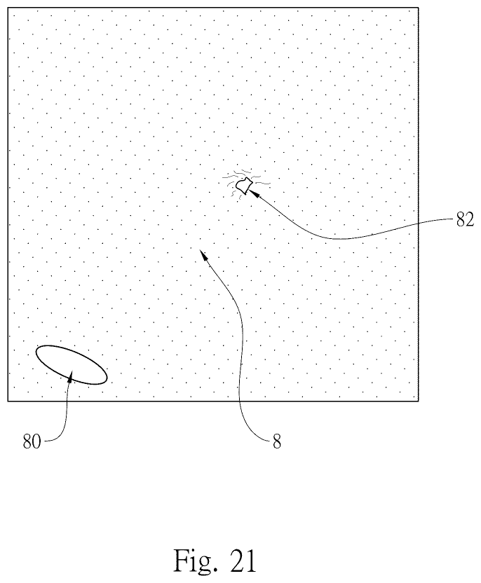

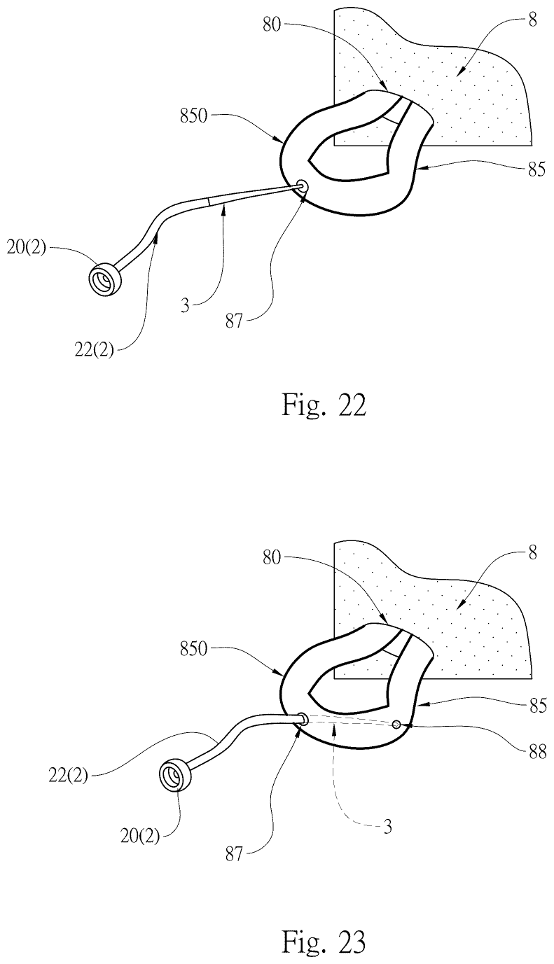

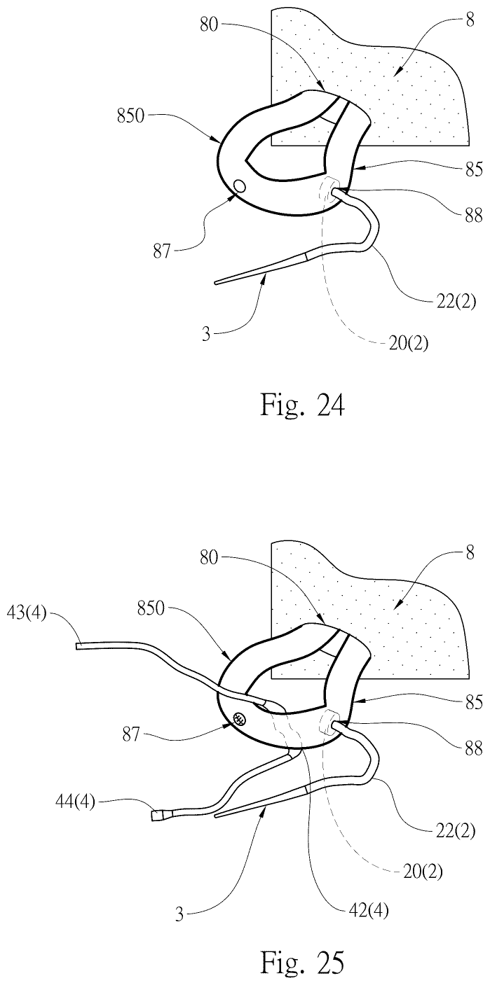

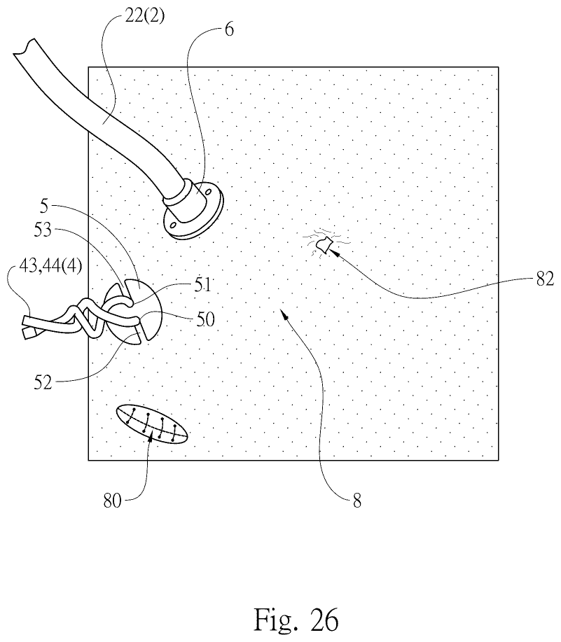

The present invention further provides a second embodiment of a surgical method using the artificial intestinal device 1 of the present invention. The artificial intestinal device 1 of the present invention comprises an artificial ileal tube 2 (elastic and flexible), a guiding member 3, a fixing member 4 (elastic and flexible), a fixing plate 5 and a fixing flange 6. The surgical method using the artificial intestinal device 1 of the present invention may be a minimally invasive surgery. The surgical method using the artificial intestinal device 1 of the present invention comprises following steps of: Step A2: (please refer to FIG. 21, which shows a schematic front view of an exterior of an abdomen wall of a patient undergoing surgery) making a first incision 80 of an abdominal wall 8 of a patient, wherein the first incision 80 of the abdominal wall 8 is around a right lower corner of an abdomen of the patient (as shown in a left lower corner of FIG. 21); Step B2: identifying a location of a terminal ileum 89 of an ileum 85 in an abdomen cavity of the patient (the terminal ileum 89 of the ileum 85 is where the ileum 85 connected to a cecum 86); Step C2: (please refer to FIG. 22, which shows a schematic front view of the exterior of the abdomen wall of the patient undergoing surgery) pulling a near terminal ileum portion 850 of the ileum 85 out of the abdominal wall 8 from the abdomen cavity of the patient through the first incision 80 of the abdominal wall 8; Step D2: making a first incision 87 of the ileum 85 in the near terminal ileum portion 850 of the ileum 85, wherein a size or an area of the first incision 87 of the ileum 85 may be approximately equal to or greater than a width W of an inflated collector portion 20 of the artificial ileal tube 2 of an assembly 10, wherein the width W of the inflated collector portion 20 of the artificial ileal tube 2 of the assembly 10 is no less than, greater than, a little bit greater than or approximately equal to an inner size of the near terminal ileum portion 850 of the ileum 85; Step E2: passing the guiding member 3 of the assembly 10 (starting from a tip portion 31 of the guiding member 3) into an interior of the ileum 85 through the first incision 87 of the ileum 85 in a direction away from the terminal ileum 89, and stop passing the guiding member 3 of the assembly 10 when the guiding member 3 of the assembly 10 is totally inserted into the interior of the ileum 85 through the first incision 87 of the ileum 85 (please refer to FIG. 23, which shows a schematic front view of the exterior of the abdomen wall of the patient undergoing surgery); i.e., the assembly 10 is inserted into the interior of the ileum 85 for about the length of the guiding member 3 (for example, about 10 cm); Step F2: making a second incision 88 of the ileum 85 in the near terminal ileum portion 850 of the ileum 85, wherein the first incision 87 of the ileum 85 is between the second incision 88 of the ileum 85 and the terminal ileum 89, wherein the second incision 88 of the ileum 85 is about, for example, a second distance away from the first incision 87 of the ileum 85, wherein the second distance may be about the length of the guiding member 3 (for example, about 10 cm); and when the guiding member 3 of the assembly 10 is totally inserted into the interior of the ileum 85 through the first incision 87 of the ileum 85, the second incision 88 of the ileum 85 is about at the location of the tip portion 31 of guiding member 3 such that the tip portion 31 of guiding member 3 is exposed to an exterior of the ileum 85 through the second incision 88 of the ileum 85; i.e., the tip portion 31 of guiding member 3 can be observed from the exterior of the ileum 85 through the second incision 88 of the ileum 85, wherein a size or an area of the second incision 88 of the ileum 85 is approximately equal to or a little bit smaller than a diameter D of the middle tube portion 22 of the artificial ileal tube 2, wherein the size or the area of the second incision 88 of the ileum 85 is smaller than the size or the area of the first incision 87 of the ileum 85 and smaller than the width W of the inflated collector portion 20 of the artificial ileal tube 2; Step G2: (please refer to FIG. 24, which shows a schematic front view of the exterior of the abdomen wall of the patient undergoing surgery) pulling the tip portion 31 of the guiding member 3 of the assembly 10 from the exterior of the ileum 85 through the second incision 88 of the ileum 85 such that the guiding member 3 of the assembly 10 is pulled out of the ileum 85 through the second incision 88 of the ileum 85 and, in the meantime, the middle tube portion 22 and the inflated collector portion 20 of the artificial ileal tube 2 of the assembly 10 are passed into the interior of the ileum 85 through the first incision 87 of the ileum 85; and continuing pulling the guiding member 3 of the assembly 10 until the middle tube portion 22 of the artificial ileal tube 2 of the assembly 10 is pulled out of the ileum 85 through the second incision 88 of the ileum 85 and the inflated collector portion 20 of the artificial ileal tube 2 is stuck at the second incision 88 of the ileum 85, such that the inflated collector portion 20 of the artificial ileal tube 2 is stayed in the interior of the ileum 85, wherein an opening of the inflated collector portion 20 of the artificial ileal tube 2 is facing to a direction away from the terminal ileum 89; Step H2: (please refer to FIG. 25, which shows a schematic front view of the exterior of the abdomen wall of the patient undergoing surgery) suturing the first incision 87 of the ileum 85; Step 12: passing one of a first end 43 of a first portion 40 and a second end 44 of a second portion 41 of the fixing member 4 through a mesentery behind the near terminal ileum portion 850 of the ileum 85 such that a cushion portion 42 of the fixing member 4 is suspended beneath the near terminal ileum portion 850 of the ileum 85; Step J2: passing the near terminal ileum portion 850 of the ileum 85, the fixing member 4 and the assembly 10 into the abdomen cavity from an exterior of the abdominal wall 8 through the first incision 80 of the abdominal wall 8; Step K2: (please refer to FIG. 16) making a third incision 83 of the abdominal wall 8 (the third incision 83 is also denoted as a for-tube-incision) and a fourth incision 84 of the abdominal wall 8 (the fourth incision 84 is also denoted as a for-fixing-incision), wherein a location of the fourth incision 84 of the abdominal wall 8 is lower than a location of the third incision 83 of the abdominal wall 8 but higher than a location of the first incision 80 of the abdominal wall 8; Step L2: passing the tip portion 31 of the guiding member 3 of the assembly 10 out of the abdominal wall 8 through the third incision 83 of the abdominal wall 8 and passing the first end 43 of the first portion 40 and the second end 44 of the second portion 41 of the fixing member 4 out of the abdominal wall 8 through the fourth incision 84 of the abdominal wall 8 respectively; Step M2: (please refer to FIG. 16) pulling the first end 43 of the first portion 40 and the second end 44 of the second portion 41 of the fixing member 4 outward from the exterior of the abdominal wall 8, and, in the meantime, pulling the tip portion 31 of the guiding member 3 of the assembly 10 outward from the exterior of the abdominal wall 8, such that the first portion 40 and the second portion 41 of the fixing member 4, the guiding member 3 and the middle tube portion 22 of the artificial ileal tube 2 are exposed to the exterior of the abdominal wall 8, and the cushion portion 42 of the fixing member 4 gradually pushes the near terminal ileum portion 850 of the ileum 85 closer to the abdominal wall 8, and continuing pulling the first portion 40 and the second portion 41 of the fixing member 4 and pulling the middle tube portion 22 of the artificial ileal tube 2 outward from the exterior of the abdominal wall 8, such that the second incision 88 of the ileum 85 is adjacent to or attached to an inner surface of the abdominal wall 8, wherein since the cushion portion 42 of the fixing member 4 is an enlarged portion (especially having a larger cross-section) and is elastic and flexible, the near terminal ileum portion 850 of the ileum 85 pushed by the enlarged cushion portion 42 of the fixing member 4 will not be hurt by local tightening; Step N2: (please refer to FIG. 26, which shows a schematic front view of the exterior of the abdomen wall of the patient undergoing surgery) passing the first portion 40 of the fixing member 4 through a first elongated notch 52 of a fixing plate 5 from a periphery of the fixing plate 5 to a first through hole 50 of the fixing plate 5 along an inverse direction of a first radially outward direction 55 and passing the second portion 41 of the fixing member 4 through a second elongated notch 53 of the fixing plate 5 from the periphery of the fixing plate 5 to a second through hole 51 of the fixing plate 5 along an inverse direction of a second radially outward direction 56, and moving the fixing plate 5 to attach to an outer surface of the abdominal wall 8, such that the first portion 40 and the second portion 41 of the fixing member 4 are fixed in the first through hole 50 and the second through hole 51 of the fixing plate 5 respectively (since a diameter of the first through hole 50 and a diameter of the second through hole 51 are smaller than a diameter of the first portion 40 and a diameter of the second portion 41 respectively; wherein the fixing member 4 is fixed from the exterior of the abdominal wall 8) and the near terminal ileum portion 850 of the ileum 85 is fixed by the cushion portion 42 of the fixing member 4, wherein the fixing plate 5 can prevent the fixing member 4 (the cushion portion 42) and the second incision 88 of the ileum 85 from slipping, shifting or moving, wherein the first portion 40 and the second portion 41 of the fixing member 4 can further be tied to secure the fixing member 4; Step O2: sleeving a fixing flange 6 on the middle tube portion 22 of the artificial ileal tube 2 and moving the fixing flange 6 to attach to the outer surface of the abdominal wall 8, such that the middle tube portion 22 of the artificial ileal tube 2 passes through a through hole 62 of the fixing flange 6 and the middle tube portion 22 of the artificial ileal tube 2 is sleeved and fixed by the through hole 62 of the fixing flange 6, wherein the fixing flange 6 may be sleeved on the middle tube portion 22 of the artificial ileal tube 2 when the guiding member 3 is connected to the middle tube portion 22 of the artificial ileal tube 2 or the fixing flange 6 may be sleeved on the middle tube portion 22 of the artificial ileal tube 2 after the guiding member 3 is detached from the middle tube portion 22 of the artificial ileal tube 2; and then passing a suture (or sutures) through two through holes 63, 64 of the fixing flange 6 and the abdominal wall 8 respectively for fixing the fixing flange 6 to the outer surface of the abdominal wall 8 more tightly by the suture (or sutures), wherein the fixing flange 6 can prevent the inflated collector portion 20 and the middle tube portion 22 of the artificial ileal tube 2 (especially a portion of the middle tube portion 22 of the artificial ileal tube 2 in the through hole 62 of the fixing flange 6) from slipping, shifting or moving; Step P2: suturing the first incision 80 of the abdominal wall 8; Step Q2: detaching the connection portion 30 of the guiding member 3 of the assembly 10 from the connection tube portion 21 of the middle tube portion 22 of the artificial ileal tube 2 of the assembly 10; and Step R2: connecting a collecting device (not shown in Figure; for example, a collecting bag or an urinary bag) to the connection tube portion 21 of the middle tube portion 22 of the artificial ileal tube 2, so that the inflated collector portion 20 of the artificial ileal tube 2 can collect an intestinal fluid from an interior of the ileum 85 and then the intestinal fluid collected by the inflated collector portion 20 of the artificial ileal tube 2 can be guided to flow from the interior of the ileum 85 to the exterior of the abdominal wall 8 to the collecting device (not shown in Figure) through the middle tube portion 22 of the artificial ileal tube 2. In current embodiment, the first incision 80, the third incision 83 (the for-tube-incision) and the fourth incision 84 (the for-fixing-incision) of the abdominal wall 8, and the first incision 87 and the second incision 88 of the ileum 85 are relatively small. The first incision 87 of the ileum 85, the first incision 80 of the abdominal wall 8 will all be sutured. The ileum 85 will not exposed to the exterior of the abdominal wall 8. Hence, the risk of infection will be significantly reduced. The near terminal ileum portion 850 of the ileum 85 is pushed by the elastic, flexible and enlarged cushion portion 42 of the fixing member 4, so that the near terminal ileum portion 850 of the ileum 85 is fixed more tightly yet will not be hurt by local tightening; hence, the fixation of the near terminal ileum portion 850 of the ileum 85 is strengthened. There is no need to perform another surgery to remove the artificial ileal tube 2 (including the inflated collector portion 20 of the artificial ileal tube 2) and the fixing member 4 (including the cushion portion 42). Since the artificial ileal tube 2 and the fixing member 4 are elastic and flexible, the fixing member 4 is capable of being pulled out of the abdominal wall 8 through the fourth incision 84 of the abdominal wall 8 and the artificial ileal tube 2 is capable of being pulled out of the ileum 85 through the second incision 88 of the ileum 85 and being pulled out of the abdominal wall 8 through the third incision 83 of the abdominal wall 8. The second incision 88 of the ileum 85 is quite small; hence, the second incision 88 of the ileum 85 can be healed naturally and does not require sutures. The third incision 83 of the abdominal wall 8 can be sutured. The fourth incision 84 of the abdominal wall 8 can be healed naturally or can be sutured. Therefore, the risk of ileostomy necrosis is significantly reduced. The connection tube portion 21 of the middle tube portion 22 of the artificial ileal tube 2 is directly connected to the collecting device (not shown in Figure). The intestinal fluid collected by the inflated collector portion 20 of the artificial ileal tube 2 will be guided to flow from the interior of the ileum 85 to the exterior of the abdominal wall 8 to the collecting device (not shown in Figure) through the middle tube portion 22 of the artificial ileal tube 2. Hence, the leakage of the intestinal fluid will be prevented.

In some embodiments, after the Step L2 and before the Step O2, the surgical method using the artificial intestinal device 1 of the present invention may further comprises a following step of: suturing the third incision 83 of the abdominal wall 8. In some embodiments, after the Step M2 and before the Step N2, the surgical method using the artificial intestinal device 1 of the present invention may further comprises a following step of: suturing the fourth incision 84 of the abdominal wall 8. In some other embodiments, the fourth incision 84 of the abdominal wall 8 can be healed naturally and does not require sutures.

The guiding member 3 is preferable to have a certain hardness, so that it is more easier for passing the guiding member 3 (starting from the tip portion 31 of the guiding member 3) into the interior of the ileum 85 through the first incision 87 of the ileum 85, and also more easier for passing the guiding member 3 (starting from the tip portion 31 of the guiding member 3) out of the ileum 85 through the second incision 88 of the ileum 85, and also more easier for passing the guiding member 3 (starting from the tip portion 31 of the guiding member 3) out of the abdominal wall 8 through the third incision 83 of the abdominal wall 8.

In some special cases, the artificial ileal tube 2 (including the inflated collector portion 20) needs to be removed from the exterior of the abdominal wall 8 (by pulling the middle tube portion 22 of the artificial ileal tube 2 outward from the exterior of the abdominal wall 8); however, the fixing member 4 does not need to be removed (i.e., the near terminal ileum portion 850 of the ileum 85 between the first incision 87 and the second incision 88 of the ileum 85 remains fixed by the cushion portion 42 of the fixing member 4 to be closer to the inner surface of the abdominal wall 8). Then, after the artificial ileal tube 2 (including the inflated collector portion 20) is removed, the protrusion portion 71 of the obturator cap 7 can be passed through the third incision 83 of the abdominal wall 8 and plugged into the second incision 88 of the ileum 85. In some embodiments, the obturator cap 7 is made of biodegradable materials, wherein after the protrusion portion 71 of the obturator cap 7 is plugged into the second incision 88 of the ileum 85 (by passed through the third incision 83 of the abdominal wall 8); and then after a period of time, the obturator cap 7 will biodegrade, such that the third incision 83 of the abdominal wall 8 and the second incision 88 of the ileum 85 will be healed. After the second incision 88 of the ileum 85 and the third incision 83 of the abdominal wall 8 are healed, the healed second incision 88 of the ileum 85 is adhered to the healed third incision 83 of the abdominal wall 8, such that the leakage of the intestinal fluid is prevented.

In some special cases, the artificial ileal tube 2 (including the inflated collector portion 20) needs to be replaced; however, the fixing member 4 does not need to be removed. After the artificial ileal tube 2 (including the inflated collector portion 20) is removed, it is possible to provide another new artificial ileal tube 2 (not shown in Figure) and insert the inflated collector portion 20 of the new artificial ileal tube 2 into the ileum 85 through the third incision 83 of the abdominal wall 8 and the second incision 88 of the ileum 85.

In some special cases, the fixing member 4 needs to be replaced; however, the artificial ileal tube 2 (including the inflated collector portion 20) no needs to be removed. The fixing member 4 can be replaced through the following steps of: providing another new fixing member 4 (not shown in Figure); connecting the first end 43 of the first portion 40 of the fixing member 4 to the second end 44 of the second portion 41 of the new fixing member 4; pulling the second end 44 of the second portion 41 of the fixing member 4 slowly, such that the whole fixing member 4 is pulled out of the abdominal wall 8 through the fourth incision 84 of the abdominal wall 8 and the second end 44 of the second portion 41 of the new fixing member 4 is also exposed to the exterior of the abdominal wall 8; pulling the second end 44 of the second portion 41 of the new fixing member 4 slowly, such that the cushion portion 42 of the new fixing member 4 is approximately suspended beneath the near terminal ileum portion 850 of the ileum 85 between the first incision 87 and the second incision 88 of the ileum 85; and then fixing the new fixing member 4 with the fixing plate 5.

In some special cases, the artificial ileal tube 2 (including the inflated collector portion 20) needs to be replaced and also the fixing member 4 needs to be replaced too.

In some embodiments, the width W of the inflated collector portion 20 may be approximately equal to or a little bit greater than a diameter of the ileum 85, so that the leakage of the intestinal fluid will be prevented.

In some embodiments, the cushion portion 42 is a hollow tube portion and the first end 45 of the cushion portion 42 is connected to and in communication with the second end 47 of the first portion 40. In some other embodiments, the cushion portion 42 is a hollow tube portion and the second end 46 of the cushion portion 42 is connected to and in communication with the first end 48 of the second portion 41. In some embodiments, the first end 45 of the cushion portion 42 tapers gradually from the cushion portion 42 to the second end 47 of the first portion 40. In some other embodiments, the second end 46 of the cushion portion 42 tapers gradually from the cushion portion 42 to the first end 48 of the second portion 41. In some embodiments, the first portion 40 of the fixing member 4 is solid (not a hollow tube), elastic and flexible. In some other embodiments, the second portion 41 of the fixing member 4 is solid (not a hollow tube), elastic and flexible. In some other embodiments, the cushion portion 42 of the fixing member 4 is solid (not a hollow tube), elastic and flexible.

In some embodiments, the surgical tools of minimally invasive surgery may be, for example, robotic arms of a da Vinci robotic surgical system or some other surgical tools, such as an electrosurgical unit (or electro knife). In some embodiments, the first incision 87 of the ileum 85 is made by an electrosurgical unit (or electro knife). In some embodiments, the second incision 88 of the ileum 85 is made by an electrosurgical unit (or electro knife). In some embodiments, the second incision 88 of the ileum 85 is made by an electrosurgical unit (or electro knife), and the guiding member 3 is made of the material that can resist the high temperature (for example 150° C.) of the electrosurgical unit (or electro knife).

The artificial ileal tube 2 must have high biocompatibility. In some embodiments, the artificial ileal tube 2 may be made of silicone, wherein an outer surface of the artificial ileal tube 2 must be anti-adhesive to tissue and cells. In some embodiments, the diameter D of the middle tube portion 22 of the artificial ileal tube 2 is greater than or equal to 7 mm and less than or equal to 10 mm. In some embodiments, the diameter D of the middle tube portion 22 of the artificial ileal tube 2 is equal to 7.5 mm and less than or equal to 8.5 mm. In some embodiments, a length of the middle tube portion 22 of the artificial ileal tube 2 is greater than or equal to 180 mm and less than or equal to 120 mm. In some embodiments, a length of the middle tube portion 22 of the artificial ileal tube 2 is greater than or equal to 140 mm and less than or equal to 160 mm. In some embodiments, the width W of the inflated collector portion 20 of the artificial ileal tube 2 is greater than or equal to 18 mm and less than or equal to 28 mm. In some embodiments, the width W of the inflated collector portion 20 of the artificial ileal tube 2 is greater than or equal to 20 mm and less than or equal to 25 mm. In some embodiments, the width W of the inflated collector portion 20 of the artificial ileal tube 2 is greater than or equal to 12 mm and less than or equal to 18 mm. In some embodiments, the width W of the inflated collector portion 20 of the artificial ileal tube 2 is greater than or equal to 14 mm and less than or equal to 16 mm. In some embodiments, a length S of the inflated collector portion 20 of the artificial ileal tube 2 is greater than or equal to 10 mm and less than or equal to 18 mm. In some embodiments, the length S of the inflated collector portion 20 of the artificial ileal tube 2 is greater than or equal to 12 mm and less than or equal to 15 mm. In some embodiments, the length S of the inflated collector portion 20 of the artificial ileal tube 2 is greater than or equal to 8 mm and less than or equal to 12 mm. In some embodiments, the length S of the inflated collector portion 20 of the artificial ileal tube 2 is greater than or equal to 9 mm and less than or equal to 11 mm. In some embodiments, the inflated collector portion 20 and the middle tube portion 22 of the artificial ileal tube 2 are one-piece molded. In some embodiments, the inflated collector portion 20 and the middle tube portion 22 of the artificial ileal tube 2 are not one-piece molded, wherein the inflated collector portion 20 may be screwed with the middle tube portion 22.

In some embodiments, the guiding member 3 may be made of plastic, silicone, stainless or ceramics. In some embodiments, the diameter of the first end 32 of the guiding member 3 is greater than or equal to 7 mm and less than or equal to 10 mm. In some embodiments, the diameter of the first end 32 of the guiding member 3 is greater than or equal to 7.5 mm and less than or equal to 8.5 mm. In some embodiments, the diameter of the second end 33 of the guiding member 3 is greater than or equal to 1 mm and less than or equal to 3 mm. In some embodiments, the diameter of the second end 33 of the guiding member 3 is greater than or equal to 1.5 mm and less than or equal to 2.5 mm. In some embodiments, a length of the guiding member 3 is greater than or equal to 90 mm and less than or equal to 140 mm. In some embodiments, a length of the guiding member 3 is greater than or equal to 110 mm and less than or equal to 130 mm. In some embodiments, the connection portion 30 of the guiding member 3 is capable of being detachably sleeved and clamped by the connection tube portion 21 of the middle tube portion 22 of the artificial ileal tube 2 to form the assembly 10. In some embodiments, the connection portion 30 of the guiding member 3 is capable of being screwed with the connection tube portion 21 of the middle tube portion 22 of the artificial ileal tube 2 to form the assembly 10.

The fixing member 4 must have high biocompatibility. In some embodiments, the fixing member 4 may be made of silicone, wherein an outer surface of the fixing member 4 must be anti-adhesive to tissue and cells. In some embodiments, the diameter of the first portion 40 and the diameter of the second portion 41 of the fixing member 4 are greater than or equal to 2.5 mm and less than or equal to 5.5 mm. In some embodiments, the diameter of the first portion 40 and the diameter of the second portion 41 of the fixing member 4 are greater than or equal to 3.5 mm and less than or equal to 4.5 mm. In some embodiments, a length L of the cushion portion 42 of the fixing member 4 is greater than or equal to 45 mm and less than or equal to 65 mm. In some embodiments, the length L of the cushion portion 42 of the fixing member 4 is greater than or equal to 47 mm and less than or equal to 55 mm. In some embodiments, the diameter of the cushion portion 42 of the fixing member 4 is greater than or equal to 4 mm and less than or equal to 10 mm. In some embodiments, the diameter of the cushion portion 42 of the fixing member 4 is greater than or equal to 5 mm and less than or equal to 7 mm. In some embodiments, a length of the fixing member 4 is greater than or equal to 260 mm and less than or equal to 360 mm. In some embodiments, the length of the fixing member 4 is greater than or equal to 290 mm and less than or equal to 330 mm.

The fixing plate 5 must have high biocompatibility. In some embodiments, the fixing plate 5 may be made of silicone, wherein an outer surface of the fixing plate 5 must be anti-adhesive to tissue and cells. In some other embodiments, the fixing plate 5 may be not a circular shape. In some embodiments, the diameter of the first through hole 50 and the diameter of the second through hole 51 of the fixing plate 5 are greater than or equal to 2 mm and less than or equal to 4.5 mm. In some embodiments, the diameter of the first through hole 50 and the diameter of the second through hole 51 of the fixing plate 5 are greater than or equal to 2.5 mm and less than or equal to 3.5 mm. In some embodiments, a diameter of the fixing plate 5 is greater than or equal to 25 mm and less than or equal to 35 mm. In some embodiments, the diameter of the fixing plate 5 is greater than or equal to 28 mm and less than or equal to 32 mm. In some embodiments, a thickness of the fixing plate 5 is greater than or equal to 0.8 mm and less than or equal to 3 mm. In some embodiments, the thickness of the fixing plate 5 is greater than or equal to 0.8 mm and less than or equal to 1.5 mm.

The fixing flange 6 must have high biocompatibility. In some embodiments, the fixing flange 6 may be made of silicone, wherein an outer surface of the fixing flange 6 must be anti-adhesive to tissue and cells. In some embodiments, the outer diameter R4 of the protrusion portion 61 of the fixing flange 6 is greater than or equal to 7 mm and less than or equal to 11 mm. In some embodiments, the outer diameter R4 of the protrusion portion 61 of the fixing flange 6 is greater than or equal to 8 mm and less than or equal to 10 mm. In some embodiments, a height T of the protrusion portion 61 of the fixing flange 6 is greater than or equal to 7 mm and less than or equal to 18 mm. In some embodiments, the height T of the protrusion portion 61 of the fixing flange 6 is greater than or equal to 10 mm and less than or equal to 15 mm. In some embodiments, the height T of the protrusion portion 61 of the fixing flange 6 is greater than or equal to 7 mm and less than or equal to 11 mm. In some embodiments, the height T of the protrusion portion 61 of the fixing flange 6 is greater than or equal to 8 mm and less than or equal to 10 mm. In some embodiments, the diameter of the through hole 62 of the fixing flange 6 is greater than or equal to 6 mm and less than or equal to 9 mm. In some embodiments, the diameter of the through hole 62 of the fixing flange 6 is greater than or equal to 6 mm and less than or equal to 8 mm. In some embodiments, a thickness of the plate 60 of the fixing flange 6 is greater than or equal to 0.8 mm and less than or equal to 3 mm. In some embodiments, the thickness of the plate 60 of the fixing flange 6 is greater than or equal to 0.8 mm and less than or equal to 1.5 mm. In some embodiments, the outer diameter R3 of the plate 60 of the fixing flange 6 is greater than or equal to 18 mm and less than or equal to 35 mm. In some embodiments, the outer diameter R3 of the plate 60 of the fixing flange 6 is greater than or equal to 22 mm and less than or equal to 32 mm. In some embodiments, the outer diameter R3 of the plate 60 of the fixing flange 6 is greater than or equal to 25 mm and less than or equal to 30 mm. In some embodiments, the outer diameter R3 of the plate 60 of the fixing flange 6 is greater than or equal to 13 mm and less than or equal to 18 mm. In some embodiments, the outer diameter R3 of the plate 60 of the fixing flange 6 is greater than or equal to 14 mm and less than or equal to 16 mm.

The obturator cap 7 must have high biocompatibility. In some embodiments, the obturator cap 7 may be made of silicone, wherein an outer surface of the obturator cap 7 must be anti-adhesive to tissue and cells. In some embodiments, the color of the obturator cap 7 may be similar to the skin color of the patient. In some embodiments, the diameter R6 of the protrusion portion 71 of the obturator cap 7 is greater than or equal to 8 mm and less than or equal to 12 mm. In some embodiments, the diameter R6 of the protrusion portion 71 of the obturator cap 7 is greater than or equal to 9 mm and less than or equal to 11 mm. In some embodiments, a height H of the protrusion portion 71 of the obturator cap 7 is greater than or equal to 17 mm and less than or equal to 23 mm. In some embodiments, the height H of the protrusion portion 71 of the obturator cap 7 is greater than or equal to 19 mm and less than or equal to 21 mm. In some embodiments, the diameter R5 of the plate 70 of the obturator cap 7 is greater than or equal to 25 mm and less than or equal to 35 mm. In some embodiments, the diameter R5 of the plate 70 of the obturator cap 7 is greater than or equal to 28 mm and less than or equal to 32 mm. In some embodiments, a thickness of the plate 70 of the obturator cap 7 is greater than or equal to 0.5 mm and less than or equal to 3 mm. In some embodiments, the thickness of the plate 70 of the obturator cap 7 is greater than or equal to 0.8 mm and less than or equal to 1.5 mm. In some embodiments, the protrusion portion 71 of the obturator cap 7 tapers gradually from the top surface 72 of the plate 70 (a bottom of the protrusion portion 71) to a top of the protrusion portion 71.

As disclosed in the above description and attached drawings, the present invention can provide an artificial intestinal device and a surgical method using the artificial intestinal device. It is new and can be put into industrial use.

Although the embodiments of the present invention have been described in detail, many modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A surgical method using an artificial intestinal device, wherein the artificial intestinal device includes an artificial ileal tube, a guiding member and a fixing member, wherein the artificial ileal tube is flexible, the artificial ileal tube includes a middle tube portion and an inflated collector portion, the inflated collector portion is disposed at one end of the middle tube portion, the inflated collector portion is in communication with the middle tube portion, a width of the inflated collector portion of the artificial ileal tube is greater than a diameter of the middle tube portion of the artificial ileal tube and a diameter of the guiding member, wherein one end of the guiding member has a tip portion, the other end of the guiding member is detachably connected to the other end of the middle tube portion of the artificial ileal tube to form an assembly, wherein the fixing member is flexible, the fixing member includes a first portion, a second portion and a cushion portion between the first portion and the second portion, the surgical method comprises following steps of:
Step A: making a first incision of an abdominal wall of a patient, wherein the first incision is around a right lower corner of an abdomen of the patient;
Step B: making a first incision of an ileum in a near terminal ileum portion of the ileum, wherein a size of the first incision of the ileum is approximately equal to or greater than the width of the inflated collector portion of the artificial ileal tube;
Step C: passing the guiding member of the assembly into an interior of the ileum through the first incision of the ileum;
Step D: making a second incision of the ileum in the near terminal ileum portion of the ileum such that the tip portion of the guiding member is capable of being observed from an exterior of the ileum through the second incision of the ileum, wherein the first incision of the ileum is between an terminal ileum and the second incision of the ileum, wherein a size of the second incision of the ileum is smaller than the width of the inflated collector portion of the artificial ileal tube, the size of the second incision of the ileum is approximately equal to or a little bit smaller than the diameter of the middle tube portion of the artificial ileal tube;
Step E: pulling the tip portion of the guiding member out of the ileum from the exterior of the ileum through the second incision of the ileum, such that the guiding member of the assembly is pulled out of the ileum through the second incision of the ileum and the artificial ileal tube of the assembly is passed into the interior of the ileum through the first incision of the ileum, and continuing pulling the guiding member until the middle tube portion of the artificial ileal tube is pulled out of the ileum through the second incision of the ileum and the inflated collector portion of the artificial ileal tube is stuck at the second incision of the ileum, such that the inflated collector portion of the artificial ileal tube is stayed in the interior of the ileum;
Step F: suturing the first incision of the ileum;
Step G: passing one of the first portion and the second portion of the fixing member through a mesentery behind the near terminal ileum portion of the ileum, such that the cushion portion of the fixing member is suspended beneath the near terminal ileum portion of the ileum;
Step H: making a for-tube-incision and a for-fixing-incision of the abdominal wall;
Step I: passing the tip portion of the guiding member out of the abdominal wall through the for-tube-incision of the abdominal wall and passing one end of the first portion and one end of the second portion of the fixing member out of the abdominal wall through the for-fixing-incision of the abdominal wall respectively;
Step J: pulling the one end of the first portion and the one end of the second portion of the fixing member and the tip portion of the guiding member outward from an exterior of the abdominal wall, such that the first portion and the second portion of the fixing member, the guiding member and the middle tube portion of the artificial ileal tube are exposed to the exterior of the abdominal wall, and the cushion portion of the fixing member gradually pushes the near terminal ileum portion of the ileum closer to the abdominal wall, and continuing pulling the first portion and the second portion of the fixing member and the middle tube portion of the artificial ileal tube outward from the exterior of the abdominal wall, such that the second incision of the ileum is adjacent to or attached to an inner surface of the abdominal wall;
Step K: fixing the fixing member from the exterior of the abdominal wall, such that the near terminal ileum portion of the ileum is fixed by the cushion portion of the fixing member; and
Step L: suturing the first incision of the abdominal wall.

2. The surgical method using the artificial intestinal device according to claim 1, wherein before the Step B, the surgical method further comprises a following step of:
making a second incision of the abdominal wall.

3. The surgical method using the artificial intestinal device according to claim 2, wherein the Step B, the Step C, the Step D, the Step E, the Step F and the Step G are performed in an abdomen cavity of the patient.

4. The surgical method using the artificial intestinal device according to claim 2, wherein after the Step A and before the Step C, the surgical method further comprises a following step of:
passing the assembly into an abdomen cavity of the patient through one of the first incision of the abdominal wall and the second incision of the abdominal wall.

5. The surgical method using the artificial intestinal device according to claim 2, wherein after the Step A and before the Step G, the surgical method further comprises a following step of:
passing the fixing member into an abdomen cavity of the patient through one of the first incision of the abdominal wall and the second incision of the abdominal wall.

6. The surgical method using the artificial intestinal device according to claim 2, wherein after the Step J, the surgical method further comprises a following step of:
suturing the second incision of the abdominal wall.

7. The surgical method using the artificial intestinal device according to claim 1, wherein after the Step A and before the Step B, the surgical method further comprises a following step of:
pulling the near terminal ileum portion of the ileum out of the abdominal wall from an abdomen cavity of the patient through the first incision of the abdominal wall.

8. The surgical method using the artificial intestinal device according to claim 7, wherein after the Step G and before the Step I, the surgical method further comprises a following step of:
passing the near terminal ileum portion of the ileum, the fixing member and the assembly into the abdomen cavity of the patient through the first incision of the abdominal wall.

9. The surgical method using the artificial intestinal device according to claim 1, wherein after the Step J, the surgical method further comprises following steps of:
detaching the guiding member from the middle tube portion of the artificial ileal tube; and
connecting a collecting device to the middle tube portion of the artificial ileal tube.

10. The surgical method using the artificial intestinal device according to claim 1, wherein the artificial intestinal device further includes a fixing flange, after the Step J, the surgical method further comprises a following step of:
Step M: sleeving the fixing flange on the middle tube portion of the artificial ileal tube and moving the fixing flange to attach to an outer surface of the abdominal wall, such that the middle tube portion of the artificial ileal tube passes through a through hole of the fixing flange and the middle tube portion of the artificial ileal tube is fixed by the through hole of the fixing flange.

11. The surgical method using the artificial intestinal device according to claim 10, wherein after the Step J, the surgical method further comprises following steps of:
Step N: detaching the guiding member from the middle tube portion of the artificial ileal tube; and
Step O: connecting a collecting device to the middle tube portion of the artificial ileal tube;
wherein the Step N is performed after the Step M and before the Step O or the Step M is performed after the Step N and before the Step O.

12. The surgical method using the artificial intestinal device according to claim 10, wherein after the middle tube portion of the artificial ileal tube is passed through the through hole of the fixing flange, the surgical method further comprises a following step of:
passing a suture through two through holes of the fixing flange and the abdominal wall respectively for fixing the fixing flange to the outer surface of the abdominal wall by the suture.

13. The surgical method using the artificial intestinal device according to claim 1, wherein the artificial intestinal device further includes a fixing plate, wherein, in the Step K, the first portion and the second portion of the fixing member are passed through two elongated notches of the fixing plate toward two through holes of the fixing plate respectively and the fixing plate is moved to attach to an outer surface of the abdominal wall, such that the first portion and the second portion of the fixing member are fixed in the two through holes of the fixing plate.

14. The surgical method using the artificial intestinal device according to claim 1, wherein the Step G is performed after the Step A and before the Step H.

15. An artificial intestinal device comprising:
a fixing member including a first portion, a second portion and a cushion portion between the first portion and the second portion, wherein the fixing member is flexible, the first portion and the second portion have a long stripe shape, wherein the cushion portion is used for being suspended beneath a near terminal ileum portion of an ileum after one of the first portion and the second portion is passed through a mesentery behind the near terminal ileum portion of the ileum;
an artificial ileal tube including a middle tube portion and an inflated collector portion, wherein the artificial ileal tube is flexible, the inflated collector portion is disposed at one end of the middle tube portion, the inflated collector portion is in communication with the middle tube portion, the middle tube portion includes a connection tube portion at the other end of the middle tube portion, wherein a width of the inflated collector portion is greater than a diameter of the middle tube portion, wherein the inflated collector portion is used for collecting an intestinal fluid from an interior of the ileum; and
a guiding member having a tip portion at one end and a connection portion at the other end, wherein the connection portion of the guiding member is detachably connected to the connection tube portion of the middle tube portion of the artificial ileal tube to form an assembly, the width of the inflated collector portion of the artificial ileal tube is greater than a diameter of the guiding member, wherein the tip portion of the guiding member is used for guiding the assembly passing into the interior of the ileum through a first incision of the ileum in the near terminal ileum portion of the ileum and then guiding the assembly except the inflated collector portion of the artificial ileal tube passing out of the ileum through a second incision of the ileum in the near terminal ileum portion of the ileum such that the inflated collector portion of the artificial ileal tube is stuck at the second incision of the ileum and such that the inflated collector portion of the artificial ileal tube is stayed in the interior of the ileum, and then guiding the tip portion of the guiding member passing out of an abdominal wall through a for-tube-incision of the abdominal wall, wherein after one end of the first portion and one end of the second portion of the fixing member are passed out of the abdominal wall through a for-fixing-incision of the abdominal wall, and the tip portion of the guiding member and the first portion and the second portion of the fixing member are pulled outward from an exterior of the abdominal wall, such that the assembly except the inflated collector portion of the artificial ileal tube are exposed to the exterior of the abdominal wall and such that the cushion portion of the fixing member gradually pushes the near terminal ileum portion of the ileum closer to the abdominal wall and the second incision of the ileum is adjacent to or attached to an inner surface of the abdominal wall, and then the fixing member is fixed from the exterior of the abdominal wall, such that the near terminal ileum portion of the ileum is fixed by the cushion portion of the fixing member, wherein the first incision of the ileum is between a terminal ileum and the second incision of the ileum, wherein a size of the first incision of the ileum is approximately equal to or greater than the width of the inflated collector portion of the artificial ileal tube, a size of the second incision of the ileum is smaller than the width of the inflated collector portion of the artificial ileal tube, the size of the second incision of the ileum is approximately equal to or a little bit smaller than the diameter of the middle tube portion of the artificial ileal tube, wherein after the connection portion of the guiding member is detached from the connection tube portion of the middle tube portion of the artificial ileal tube, the intestinal fluid collected by the inflated collector portion of the artificial ileal tube is guided to flow from the interior of the ileum to the exterior of the abdominal wall through the middle tube portion of the artificial ileal tube.

16. The artificial intestinal device according to claim 15, further comprising a fixing flange, wherein the fixing flange is used for sleeving on the middle tube portion of the artificial ileal tube and then the fixing flange is moved to attach to an outer surface of the abdominal wall, such that the middle tube portion of the artificial ileal tube passes through a through hole of the fixing flange and the middle tube portion of the artificial ileal tube is fixed by the through hole of the fixing flange.

17. The artificial intestinal device according to claim 16, wherein the fixing flange further comprises a fixing structure formed on an inner surface of the through hole of the fixing flange for preventing the middle tube portion of the artificial ileal tube from slipping.

18. The artificial intestinal device according to claim 15, further comprising a fixing plate, wherein the fixing plate has two through holes and two elongated notches, one of the two elongated notches extends from one of the two through holes to a periphery of the fixing plate along one direction from the one of the two through holes toward the periphery of the fixing plate, the other one of the two elongated notches extends from the other one of the two through holes to the periphery of the fixing plate along an inverse direction of the one direction, wherein a diameter of each of the two elongated notches is smaller than a diameter of each of the two through holes, the diameter of each of the two through holes is smaller than a diameter of the first portion and a diameter of the second portion of the fixing member, wherein after the first portion and the second portion of the fixing member are passed through the two elongated notches of the fixing plate toward the two through holes of the fixing plate respectively and the fixing plate is moved to attach to an outer surface of the abdominal wall, such that the first portion and the second portion of the fixing member are fixed in the two through holes of the fixing plate so as to fix the fixing member from the exterior of the abdominal wall.

19. The artificial intestinal device according to claim 18, wherein the one end of the second portion of the fixing member further includes a through hole, wherein after the first portion and the second portion of the fixing member are fixed in the two through holes of the fixing plate, the one end of the first portion of the fixing member is passed through the through hole of the one end of the second portion of the fixing member, such that the fixing member is fixed more tightly.

20. The artificial intestinal device according to claim 15, wherein the cushion portion of the fixing member includes a plurality of sub-portions, a diameter of a junction between any two adjacent sub-portions of the plurality of sub-portions is smaller than a diameter of each of the two adjacent sub-portions.

21. The artificial intestinal device according to claim 15, further comprising a obturator cap, wherein after the artificial ileal tube is pulled out of the ileum through the second incision of the ileum and out of an abdomen cavity through the for-tube-incision of the abdominal wall, a protrusion portion of the obturator cap is capable of being passed through the for-tube-incision of the abdominal wall and plugged into the second incision of the ileum, wherein the obturator cap is made of biodegradable materials for assisting the second incision of the ileum and the for-tube-incision of the abdominal wall healing.
